SEQUENCE LISTING (4 sequences)

SEQ ID NO: 1 - protein, 8 aa, Mus musculus
KAVYFATM

SEQ ID NO: 2 - protein, 8 aa, Gallus gallus
SIINFEKL

SEQ ID NO: 3 - protein, 9 aa, unidentified
  source 1..9: note = Homo sapiens and/or Mus Musculus
SVYDFFVWL

SEQ ID NO: 4 - protein, 8 aa, Vesicular stomatitis
RGYVYQGL

(19) United States
(12) Patent Application Publication
(10) Pub. No.: US 2023/0158142 A1
(43) Pub. Date: May 25, 2023

(54) BLOCKADE OF IFN SIGNALING DURING CANCER VACCINATION

(71) Applicant: McMaster University, Hamilton (CA)

(72) Inventors: Yonghong Wan, Ancaster (CA); Scott Walsh, Guelph (CA); Nader El-Sayes, Oakville (CA)

(21) Appl. No.: 17/990,480

(22) Filed: Nov. 18, 2022

Related U.S. Application Data
(60) Provisional application No. 63/281,384, filed on Nov. 19, 2021.

(51) Int. Cl. A61K 39/395; C12N 15/86; A61K 39/00; A61P 35/00; A61P 37/04

(52) U.S. Cl. CPC A61K 39/3955; C12N 15/86; A61K 39/0011; A61P 35/00; A61P 37/04; C12N 2760/20222; C12N 2760/20234; C12N 2760/20243; A61K 2039/505; A61K 2039/5256

(57) ABSTRACT

The present disclosure relates to a method for stimulating tumor antigen-specific immune responses comprising cancer vaccination involving the blockade or inhibition of interferon signaling. In particular, the method comprises administering an inhibitor of interferon signaling and a cancer vaccine comprising a tumor antigen, wherein the inhibitor of interferon signaling is administered before the cancer vaccine.

### CROSS-REFERENCE TO RELATED APPLICATIONS

This application claims the benefit of priority to U.S. Provisional Application No. 63/281,384, filed Nov. 19, 2021, the contents of which are incorporated herein by reference in their entirety.

### INCORPORATION OF SEQUENCE LISTING

A computer readable form of the Sequence Listing “3244-P66686US01_SequenceListing.xml” (4,636 bytes) created on Jan. 9, 2023, is herein incorporated by reference.

### FIELD

The present disclosure relates to the field of cancer immunotherapy, and in particular, to a method for stimulating tumor antigen-specific immune responses and/or cancer vaccination involving the blockade or inhibition of interferon signaling.

### BACKGROUND

Cancer vaccines are emerging as frontline therapeutics designed to stimulate T cell responses with a discrete tumor specificity and cytotoxic function¹. However, tumor levied immunosuppression is a prohibiting barrier to therapeutic efficacy, blunting the magnitude and function of vaccine induced responses². Thus, improved strategies are needed to enhance the potency and efficacy of cancer vaccines, especially in more suppressive and treatment refractory tumor types.

Currently, cancer vaccination strategies intentionally induce inflammation, with the general assumption that it enhances vaccination effects³. Interferon, both type 1 interferon (T1IFN) and type 2 interferon (T2IFN or IFNγ), represent major components of the inflammatory profile of cancer vaccines^{4,5}. These are pleiotropic cytokines with important roles in innate antiviral immunity, although T2IFN is also viewed as an effector cytokine since its expression is induced in activated T and natural killer (NK) cells⁶. T1IFN and T2IFN signaling induces an anti-proliferative and pro-apoptotic state in infected cells to interfere with virus replication; a function from which their name is derived^{7,8}. However, both have broader effects in regulating T cell responses to vaccination, promoting antigen-specific responses of CD8 T cells via increased expression of major histocompatibility complex class I (MHCI) and co-stimulatory signals on antigen presenting cells as well as T cell intrinsic proliferative and survival signals⁹⁻¹¹. It follows that genetic defects in IFN signaling have been shown to compromise antiviral T cell responses and tumor therapy in mouse models^{12,13}. Thus, many cancer vaccines are designed to intentionally engage these pathways, using toll-like receptor (TLR) agonists or encoding IFN directly^{3,14}. However, data has recently emerged indicating that the benefit of T1IFN for T cell responses is restricted to primary T cell responses, with no significant detriment observed when the T1IFN receptor (or interferon-α/β receptor; IFNAR) is lacking during secondary antigen stimulation (boosting) in a LCMV infection model¹⁵. Furthermore, recent reports have identified a conflicting role of IFN signaling in tumor immunotherapy and tumor immunosuppression. Chronic IFN signaling has been shown to serve as a mechanism behind immunosuppression in the tumor microenvironment and as a mechanism of escape from checkpoint inhibitor blockade therapy (CIB)¹⁶⁻¹⁸. As well, a detrimental role for T1IFN has been demonstrated for adoptive T cell therapy (ACT), promoting attrition and truncating persistence of transferred chimeric antigen receptor (CAR) T cells¹⁹.

Antigen-experienced T cells arising during tumor growth dominate the response and clinical effect in cancer immunotherapies²⁰⁻²³. Nevertheless, priming of T cells under the influence of tumor levied immunosuppressive mechanisms (tumor priming) often renders these cells dysfunctional²⁴. Currently, cancer immunotherapies, such as CIB and tumor infiltrating lymphocyte (TIL)-based ACT, strive to make use of tumor-primed T cells by inhibiting specific inhibitory ligand signals or growing them ex vivo in the absence of tumor interference²⁵. These methods address some of the deficiencies in cancer immunotherapy, such as low magnitudes of tumor-specific T cells or inhibition of their effector function in the tumor microenvironment (TME), but are insufficient to show clinical effect in more suppressive and treatment resistant tumor types²⁶. Thus, cancer vaccines with the capacity to simultaneously augment tumor-specific immunity and modulate immunosuppression in the TME are a desirable treatment modality.

Owing to their preferential replication in tumor cells and multimodal effects on the immune system, oncolytic viruses (OVs) are a uniquely suited vector for cancer vaccination. Initial oncolysis supports the stimulation of tumor-specific T cell responses via loading of antigen presenting cells in the tumor and draining lymph nodes with antigens from lysed tumor cells²⁷. Furthermore, antigenic stimulation can be augmented by engineering antigen coding capacity into OVs, simultaneously stimulating T cell responses, recruiting T cells to the tumor bed, and supporting their function^{27,28}. Thereby, OVs have a promising role as cancer vaccines due their distinct capacity to stimulate anti-tumor immunity with local and systemic inflammation supportive to tumor attack.

### SUMMARY

Interferon is potently induced by many cancer vaccines, such as OV vectors²⁸ and the present inventors have demonstrated a beneficial effect of modulating signaling of this component of a vaccine that comprises a tumor antigen.

Accordingly, the present disclosure provides a method for stimulating tumor antigen-specific immune responses, the method comprising administering an inhibitor of interferon (IFN) signaling and a cancer vaccine comprising a tumor antigen to a subject in need thereof, wherein the inhibitor of IFN is administered before the cancer vaccine.

In some embodiments, the method is for treating cancer in the subject.

In some embodiments, the cancer is selected from the group consisting of melanoma, sarcoma, lymphoma, carcinoma, brain cancer (e.g. glioma), breast cancer, liver cancer, lung cancer, kidney cancer, pancreatic cancer, esophageal cancer, stomach cancer, colon cancer, colorectal cancer, bladder cancer, prostate cancer and leukemia.

In some embodiments, the inhibitor is administered from about 5 minutes to about 48 hours before the cancer vaccine is administered.

In some embodiments, the inhibitor is administered from about 30 minutes to about 24 hours before the cancer vaccine is administered.

In some embodiments, the inhibitor is administered from about 2 hours to about 4 hours before the cancer vaccine is administered.

In some embodiments, the interferon comprises type 1 interferon (T1IFN) and/or type 2 interferon (T2IFN).

In some embodiments, the inhibitor comprises an antibody, or fragment thereof, that specifically binds and neutralizes IFNα and/or IFNβ.

In some embodiments, the inhibitor comprises an antibody, or fragment thereof, that specifically binds and blocks a T1IFN receptor.

In some embodiments, the inhibitor is an antibody, or fragment thereof, that binds and neutralizes IFNγ.

In some embodiments, the inhibitor is an antibody, or fragment thereof, that specifically binds and blocks a T2IFN receptor.

In some embodiments, the cancer vaccine comprises an oncolytic virus that expresses a tumor antigen.

In some embodiments, the oncolytic virus is selected from the group consisting of Herpesviridae, Rhabdoviridae, Picornaviradae, Reoviridae, Togaviridae, Adenoviridae, Paramyxoviridae, and Poxviridae.

In some embodiments, the oncolytic virus is a rhabdovirus.

In some embodiments, the rhabdovirus is a recombinant or wildtype vesicular stomatitis virus.

In some embodiments, the cancer vaccine and/or the inhibitor is administered by injection.

In some embodiments, the tumor antigen is a tumor-associated antigen and/or a tumor-specific antigen.

In some embodiments, the tumor-associated antigen is selected from the group consisting of alphafetoprotein (AFP), carcinoembryonic antigen (CEA), CA 125, Her2, dopachrome tautomerase (DCT), GP100, Melan-A/MART-1, MAGE proteins, BAGE proteins, GAGE proteins, NY-ESO1, WT-1, survivin, tyrosinase, SSX2, Cyclin-A1, KIF20A, MUC5AC, Meloe, Lengsin, Kallikrein 4, IGF2B3, glypican-3, HPV E6 and HPV E7.

In some embodiments, the method further comprises administering adoptive cell transfer (ACT) cells and/or a checkpoint inhibitor.

Also provided herein is a kit comprising an inhibitor of interferon (IFN) signaling, a cancer vaccine comprising a tumor antigen, and instructions for use of the kit.

### DRAWINGS

Certain embodiments of the disclosure will now be described in greater detail with reference to the attached drawings in which:

FIG. 1A-F shows that IFNAR blockade enhances tumor-specific responses derived from endogenous T cells with no concurrent effect on transferred central memory T cells in exemplary embodiments of the disclosure. B16gp33 tumor bearing mice were treated with VSV-gp33 and IFNAR blockade according to the schematic shown in FIG. 1A. Antigen-specific T cell responses were quantified by assessing the total CD8+ T cells producing IFNγ⁺ in response to gp33 peptide stimulation with the results separated based upon Thy1.1 staining in FIG. 1B (Thy1.1⁺ responses are derived from transferred P14T_{CM} cells and Thy1.1⁻ responses derived from endogenous T cells). (FIG. 1C) Representative flow plots showing IFNγ and TNFα expression of transferred cells (Thy1.1⁺) and endogenous T cells (Thy1.1⁻) after vaccination with the indicated treatment. (FIG. 1D) Tumor response curves are also shown. (FIG. 1E) Quantification of responses generated by combining IFNAR blockade with VSV vaccination and T_{CM} T cell transfer targeting a different antigen (VSV-DCT and 24H9 T_{CM}). (FIG. 1F) Quantification of responses generated by combining IFNγ neutralizing antibody with VSV-gp33 vaccination and P14 T_{CM} transfer treating B16gp33 tumors.

FIG. 2A-K shows that IFNAR blockade enhances tumor-specific responses derived from endogenous T cells in the absence of transferred cells in exemplary embodiments of the disclosure. (FIG. 2A) Tumor volume and (FIG. 2B) gp33 peptide-specific response in the circulation of B16gp33 tumor bearing mice after vaccination with the VSV-gp33 alone or in combination with IFNAR blockade as quantified by IFNγ expression detection in response to peptide stimulation of blood samples. IFNAR blockade alone and PBS treatment controls are also shown. (FIG. 2C) Survival of mice after treatment was compared between the VSV-gp33 treated and VSVO-gp33+IFNAR blockade treated groups. (FIG. 2D) Tumor growth curves and (FIG. 2E) gp33-specific T cell responses in the circulation of B16gp33 tumor bearing mice treated VSV-GFP (an antigen negative control virus) or VSV-GFP in combination with IFNAR blockade to control for the effect of virus replication in the absence of a direct vaccine effect. (FIG. 2F) Quantification of SVY (immunodominant epitope of DCT/TRP-2) peptide-specific responses generated by VSV-DCT vaccination alone or in combination with IFNAR blockade of B16gp33 tumor bearing mice. (FIG. 2G) Gp33-specific T cell responses in the circulation of B16gp33 tumor bearing mice after intratumoral treatment with VSV-gp33 alone or with VSV-gp33 in combination with IFNAR blockade. (FIG. 2H) Quantification of gp33-specific T cell responses induced in the circulation of B16gp33 tumor bearing mice after VSV-gp33 vaccination, as well as tumor growth curves (FIG. 2I), showing that the effect of IFNAR blockade on response to vaccination can be partially recapitulated by antibody-based neutralization of either IFNα or IFNβ singularly and fully recapitulated when IFNα and IFNβ are both neutralized. (FIG. 2J) Quantification of gp33-specific T cell responses, as well as tumor growth curves (FIG. 2K), induced in B16gp33 tumor bearing mice after VSV-gp33 vaccination alone and when combined with IFNGR blockade pretreatment.

FIG. 3A-D demonstrates that IFNAR blockade augmentation of VSV induced responses is not antigen restricted in exemplary embodiments of the disclosure. (FIG. 3A) Tumor regression curve and gp33-specific T cell response of MO5 tumor bearing mice after treatment broken down by percent (FIG. 3B) and number (FIG. 3D). (FIG. 3C) Quantification of total CD8 T cell compartment in PBMC samples from treated mice.

FIG. 4A-D shows that IFNAR blockade augments VSV induced responses in other tumor models in exemplary embodiments of the disclosure. (FIG. 4A and FIG. 4C) Tumor regression curve and (FIG. 4B and FIG. 4D) gp33-specific T cell response of MC38gp33 and MCA205gp33 tumor bearing mice after treatment, respectively.

FIG. 5A-E demonstrates that VSV induced responses are predominated by a secondary stimulation of cells primed by cross-presentation during tumor growth in exemplary embodiments of the disclosure. Gp33 responses in the circulation seven days after implantation with the indicated tumor (just prior to treatment) (FIG. 5A) or 5 days after treatment in mice with the indicated tumor (FIG. 5B). Gp33-specific responses in PBMC samples from MC38gp33 tumor bearing BATF3^{-/-} mice five days after injection of the indicated treatment (FIG. 5C). VSV-specific responses to the RGY peptide in treated B16gp33 tumor bearing mice (FIG. 5D) and gp33-specific responses in similarly treated tumor free mice (FIG. 5E).

FIG. 6A-C shows that IFNAR blockade enhances virus replication and persistence in exemplary embodiments of the disclosure. Luminescence of MO5 (B16 cells engineered to express OVA) tumor bearing mice was evaluated via D-Luciferin injection at the indicated time points after treatment with VSV-SIINFEKL-Luciferase with or without IFNAR blockade using an IVIS imaging system. Dorsal (FIG. 6A) and ventral (FIG. 6B) views shown. (FIG. 6C) Immunohistochemistry of tumor tissues harvested from B16gp33 tumor bearing mice at the indicated timepoint after treatment and probed with an antibody raised against VSV. Images are 10× magnification.

FIG. 7 demonstrates that IFNAR blockade enhances tumor infiltration of T cells in an exemplary embodiment of the disclosure. Images of tumor tissues taken from treated mice at the indicated time point after infection (days post infection or dpi) and probed for CD8 expression via immunohistochemical staining are shown. Images of 5× magnification are shown.

FIG. 8A-E shows that IFNAR blockade enhances virus replication in the tumor and lymphoid organs and prolongs antigen presentation in exemplary embodiments of the disclosure. Plaque assay quantification of infectious VSV in homogenates of (FIG. 8A) tumor, (FIG. 8B) spleen, (FIG. 8C) tumor draining lymph node (TdLN) and (FIG. 8D) non-tumor draining lymph node (nTdLN) tissues taken at the indicated time points after treatment with VSV-gp33 or VSV-gp33+αIFNAR blockade are shown. (FIG. 8E) Antigen presentation occurring as a result of VSV-gp33 or VSV-gp33+αIFNAR blockade treatment was assessed in the tissues indicated. The proportion of adoptively transferred naïve P14 T cells experiencing at least one division as determined by dilution of violet proliferation dye is shown as a representation of antigen stimulation induced proliferation (Mixed-effects model analysis).

FIG. 9A-B shows that the IFNAR blockade augmentation is specific the response of tumor-primed T cells to vaccination and does not augment responses derived from T cells primed by a previous virus infection in exemplary embodiments of the disclosure. (FIG. 9A) A schematic of the experimental procedure is shown. CD8 T cells isolated from splenocytes of a LCMV immune Thy1.1⁺ mouse (1 month post LCMV infection) using a CD8 negative selection kit were transferred intravenously to B16gp33 tumor bearing mice one day prior to VSV-gp33 vaccination. (FIG. 9B) Gp33-specific T cell responses were quantified and separated based upon Thy1.1 expression to show that responses derived from virus primed cells (Thy1.1⁺) were unchanged by IFNAR blockade while tumor-primed T cell responses (Thy1.1⁻) were significantly augmented.

FIG. 10A-H demonstrates that IFNAR blockade enhances proliferation of tumor-primed cells in response to VSV vaccination in exemplary embodiments of the disclosure. (FIG. 10A) Thy1.1⁺ naïve P14 T cells were transferred one day prior to implantation of B16gp33 tumor cells and subsequently recovered from the tumor draining lymph nodes seven days later before staining for classic markers of memory phenotype via flow cytometry. (FIG. 10B) The total number of Thy1.1⁺P14 T cells was also quantified before vaccination and at the indicated time points after treatment. (FIG. 10C) CaspGlow staining of similarly generated tumor-primed Thy1.1⁺P14 CD8 T cells one day after the indicated treatment show no change in apoptosis as indicated by caspase cleavage activity in these cells. (FIG. 10D) Ki67 staining of tumor-primed Thy1.1⁺CD8⁺P14 T cells (derived from naïve cells transferred before B16gp33 tumor implantation) recovered from tumor draining lymph nodes three days after vaccination and probed using flow cytometry. (FIG. 10E) Thy1.1⁺P14 cells recovered from the tumor draining lymph nodes of B16gp33 tumor bearing mice (tumor-primed T cells) were labeled with proliferation dye before transfer to tumor free mice. Dye dilution was evaluated by flow cytometry of Thy1.1⁺ cells in circulation five days after vaccination, which was augmented with IFNAR blockade. A proliferation model was fitted, showing an increase in the proliferation index (FIG. 10F), percent divided (FIG. 10G) and division index (FIG. 10H) with IFNAR blockade.

FIG. 11 shows that IFNAR blockade inhibits virus-induced upregulation of PD-L1 expression in the tumor in exemplary embodiments of the disclosure. Immunohistochemistry staining for PD-L1 expression on MC38 and B16F10 tumor tissues (implanted subcutaneously and intradermally, respectively) extracted one day after administering the indicated treatment. Images of the whole tumor tissue section are shown with a higher magnification of the area denoted by a black box shown below (5× magnification).

FIG. 12A-E demonstrates that IFNAR blockade partially suppresses virus-mediated upregulation of PD-L1 in circulating leukocytes in exemplary embodiments of the disclosure. (FIG. 12A) Representative histograms and (FIG. 12B) graph of PD-L1 staining intensity on total leukocytes (CD45⁺ cells) as well as graphs of similar staining in (FIG. 12C) B cells (CD19⁺ cells), (FIG. 12D) macrophages (CD11b⁺F4/80⁺) and (FIG. 12E) monocytes (Ly6C⁺Ly6G⁻) extracted from venous blood samples taken 24 hours after the indicated treatment.

FIG. 13A-C shows that PD-L1 blockade has redundant effect with IFNAR blockade when combined with VSV vaccination and fails to improve therapeutic outcomes in exemplary embodiments of the disclosure. (FIG. 13A) Regression curves and (FIG. 13B) gp33-specific T cell response quantification of B16gp33 tumor bearing mice treated with the indicated treatment. (FIG. 13C) Resultant Kaplan-Meier survival curve graph.

FIG. 14A-C shows that CTLA4 blockade is compatible when combined with VSV vaccination and IFNAR blockade and works to suppress tumor relapse in exemplary embodiments of the disclosure. (FIG. 14A) Tumor regression curves of B16gp33 tumors when treated with VSV-gp33 as well as the indicated antibody treatment with the resultant survival plots (FIG. 14B) and (FIG. 14C) gp33-specific T cell response quantification.

### DETAILED DESCRIPTION

### I. Definitions

Unless otherwise indicated, the definitions and embodiments described in this and other sections are intended to be applicable to all embodiments and aspects of the present disclosure herein described for which they are suitable as would be understood by a person skilled in the art. It is also to be understood that the terminology used herein is for the purpose of describing particular aspects only and is not intended to be limiting.

In understanding the scope of the present application, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. The term “consisting” and its derivatives, as used herein, are intended to be closed terms that specify the presence of the stated features, elements, components, groups, integers, and/or steps, but exclude the presence of other unstated features, elements, components, groups, integers and/or steps. The term “consisting essentially of”, as used herein, is intended to specify the presence of the stated features, elements, components, groups, integers, and/or steps as well as those that do not materially affect the basic and novel characteristic(s) of features, elements, components, groups, integers, and/or steps.

Terms of degree such as “substantially”, “about” and “approximately” as used herein mean a reasonable amount of deviation of the modified term such that the end result is not significantly changed. These terms of degree should be construed as including a deviation of at least 5% of the modified term if this deviation would not negate the meaning of the word it modifies. The modifier “about” used in connection with a quantity is inclusive of the stated value and has the meaning dictated by the context (e.g., it includes the degree of error associated with measurement of the particular quantity). In addition, all ranges disclosed herein are inclusive of the endpoints and also any intermediate range points, whether explicitly stated or not, and the endpoints are independently combinable with each other.

As used in this application, the singular forms “a”, “an” and “the” include plural references unless the content clearly dictates otherwise. For example, an embodiment including “a T cell” should be understood to present certain aspects with one T cell or two or more additional T cells.

In embodiments comprising an “additional” or “second” component, such as an additional or second vaccine, the second component as used herein is chemically different from the other components or first component. A “third” component is different from the other, first, and second components, and further enumerated or “additional” components are similarly different.

The term “and/or” as used herein means that the listed items are present, or used, individually or in combination. In effect, this term means that “at least one of” or “one or more” of the listed items is used or present.

The term “or” as used herein is intended to include “and” unless the context clearly indicates otherwise

The abbreviation, “e.g.” is derived from the Latin exempli gratia and is used herein to indicate a non-limiting example. Thus, the abbreviation “e.g.” is synonymous with the term “for example.”

The term “treating”, “treatment”, and the like, as used herein, and as is well understood in the art, refers to an approach for obtaining beneficial or desired results, including clinical results. Beneficial or desired clinical results include, but are not limited to alleviation or amelioration of one or more symptoms or conditions, arresting development of disease, diminishment of extent of disease, stabilized (i.e. not worsening) state of disease, preventing spread of disease, delay or slowing of disease progression, amelioration or palliation of the disease state, including regression of the disease, diminishment of the reoccurrence of disease, and remission (whether partial or total), whether detectable or undetectable. “Treating” and “treatment” may also refer to prolonging survival as compared to expected survival if not receiving treatment. “Treating” and “treatment” as used herein also include prophylactic treatment. The effect may be prophylactic in terms of completely or partially preventing a disease or symptom thereof and/or may be therapeutic in terms of affecting a partial or complete cure for a disease and/or symptoms of the disease. For example, a subject with early cancer can be treated to prevent progression, or alternatively a subject in remission can be treated to prevent recurrence. Prophylactic treatment includes preventing the disease or a symptom of a disease from occurring in a subject which may be predisposed to the disease but has not yet been diagnosed as having it (e.g., including diseases that may be associated with or caused by a primary disease).

Treating may refer to any indicia of success in the treatment or amelioration or prevention of a cancer, including any objective or subjective parameter such as abatement; remission; diminishing of symptoms; or making the disease condition more tolerable to the patient; slowing in the rate of degeneration or decline; or making the final point of degeneration less debilitating. The treatment or amelioration of symptoms is based on one or more objective or subjective parameters; including the results of an examination by a physician. Accordingly, the term “treating” includes the administration of the methods of the present disclosure to prevent, delay, alleviate, arrest or inhibit development of the symptoms or conditions associated with diseases (e.g. cancer).

The term “cancer” as used herein refers to cellular-proliferative disease states.

The term “subject” as used herein includes all members of the animal kingdom including mammals such as a mouse, a rat, a dog and a human.

The term “cancer vaccination” or “cancer vaccine” as used herein refers to a composition that is capable of being administered to a subject and which induces an immune response to prevent, ameliorate or otherwise treat an infection and/or disease state and/or to reduce at least one symptom of an infection and/or disease state. Typically, on introduction to a subject, the vaccine is able to provoke cellular immunity responses including, but not limited to, stimulating T cell responses with cytotoxic function and discrete tumor specificity against a discrete antigen encoded, or otherwise provided, by the vaccine.

The term “administering” or “administration” as used herein refers to the placement of an agent, a drug, a compound, a pharmaceutical composition, an inhibitor or a vaccine as disclosed herein into a subject by a method or route which results in at least partial delivery to a desired site. The compounds and compositions disclosed herein can be administered by any appropriate route which results in an effective treatment in the subject. Possible routes of administration of the compounds and pharmaceutical compositions disclosed herein include, but are not limited to, intravenous, intraperitoneal, intramuscular, subcutaneous, transdermal, oral, buccal, sublingual, intranasal, or rectal routes of administration, or a combination thereof.

The term “antibody” as used herein refers to an immunoglobulin molecule capable of specific binding to a target through at least one antigen recognition site, located in the variable region of the immunoglobulin molecule. The antibody may be from recombinant sources and/or produced in transgenic animals, and includes, without limitation, monoclonal antibodies, chimeric and humanized antibodies, and binding fragments thereof, including for example a single chain Fab fragment, Fab′2 fragment, or single chain Fv fragment. There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2. Humanized or other chimeric antibodies may include sequences from one or more than one isotype, class, or species.

The basic antibody structural unit is known in the art to comprise a tetramer composed of two identical pairs of polypeptide chains, each pair having one light (“L”) (about 25 kDa) and one heavy (“H”) chain (about 50-70 kDa). The amino-terminal portion of the light chain forms a light chain variable domain (VL) and the amino-terminal portion of the heavy chain forms a heavy chain variable domain (VH). Together, the VH and VL domains form the antibody variable region (Fv) which is primarily responsible for antigen recognition/binding. Within each of the VH and VL domains are three hypervariable regions or complementarity determining regions (CDRs, commonly denoted CDR-H1, CDR-H2, CDR-H3, CDR-L1, CDR-L2, and CDR-L3). The carboxy-terminal portions of the heavy and light chains together form a constant region primarily responsible for effector function. Further, these antibodies are typically produced as antigen binding fragments such as Fab, Fab′ F(ab′)2, Fd, Fv and single domain antibody fragments, or as single chain antibodies (e.g. scFv) in which the heavy and light chains are linked by a spacer or linker. The antibodies may include sequences from any suitable species including human. Also, the antibodies may exist in monomeric or polymeric form.

The term “antibody fragment” or “binding fragment” as used herein is intended to include without limitations Fab, Fab′, F(ab′)2, scFab, scFv, dsFv, ds-scFv, dimers, minibodies, diabodies, and multimers thereof, and Domain Antibodies. Antibodies can be fragmented using conventional techniques. For example, F(ab′)2 fragments can be generated by treating the antibody with pepsin. The resulting F(ab′)2 fragment can be treated to reduce disulfide bridges to produce Fab′ fragments. Papain digestion can lead to the formation of Fab fragments. Fab, Fab′ and F(ab′)2, scFv, dsFv, ds-scFv, dimers, minibodies, diabodies, and other fragments can also be synthesized by recombinant techniques.

The term “antigen” as used herein refers to a molecule containing one or more epitopes (either linear, conformational or both) that will stimulate a host's immune system to make a humoral and/or cellular antigen-specific response. The term is used interchangeably with the term “immunogen.” The term includes polypeptides which include modifications, such as deletions, additions and substitutions (generally conservative in nature) as compared to a native sequence, as long as the protein maintains the ability to elicit an immunological response, as defined herein. These modifications may be deliberate, as through site-directed mutagenesis, or may be accidental, such as through mutations of hosts which produce the antigens.

The term “epitope” as commonly used means a subunit or fragment of an antigen or immunogen that is specifically recognized by the immune system. The term includes a T-cell epitope, such as a CTL epitope, will include at least about 7-9 amino acids, and a helper T-cell epitope at least about 12-20 amino acids. The term also includes an antibody binding site, typically a polypeptide segment having a particular structural conformation, in an antigen that is specifically recognized by the antibody. Normally, a B-cell epitope will include at least about 5 amino acids but can be as small as 3-4 amino acids. Normally, an epitope will include between about 7 and 22 amino acids, inclusive, such as, 7, 8, 9, 10, 11, 12, 13, 14, 15, 16, 17, 18, 19, 20, 21, or 22 amino acids.

### II. Methods and Kits of the Disclosure

The present disclosure describes a method for stimulating tumor antigen-specific immune responses and/or for treating cancer in a mammal (e.g. human) in which vaccination against a tumor antigen is combined with an inhibitor of interferon signaling. Accordingly, in an aspect of the present disclosure, provided is a method for stimulating tumor antigen-specific immune responses, the method comprising administering an inhibitor of interferon (IFN) signaling and a cancer vaccine comprising a tumor antigen to a subject in need thereof, wherein the inhibitor of interferon is administered before the cancer vaccine.

In some embodiments, the method is for treating cancer in the subject.

In some embodiments, the cancer is melanoma, sarcoma, lymphoma, carcinoma, brain cancer (e.g. glioma), breast cancer, liver cancer, lung cancer, kidney cancer, pancreatic cancer, esophageal cancer, stomach cancer, colon cancer, colorectal cancer, bladder cancer, prostate cancer or leukemia. In some embodiments, the cancer is melanoma, fibrous sarcoma, lung carcinoma or colon carcinoma. In some embodiments, the cancer is pancreatic cancer, lung cancer or triple-negative breast cancer.

In an embodiment, the cancer is one involving more suppressive and treatment refractory tumor types. In another embodiment, the cancer is one involving tumors that induce T cell senescence.

Administration will depend on the pharmacokinetics of the inhibitor and the cancer vaccine in the presence of each other and can include administering the inhibitor from about a few minutes before the cancer vaccine, or even administering the inhibitor about two days before the cancer vaccine, if the pharmacokinetics are suitable. In some embodiments, the inhibitor is administered as a single dose before administering the cancer vaccine to provide a transient effect.

In some embodiments, the inhibitor is administered from about 5 minutes to about 48 hours before the cancer vaccine is administered, such as from about 5 minutes, about 30 minutes, about 1 hour, about 2 hours, about 3 hours, about 4 hours, about 5 hours, about 6 hours, about 9 hours, about 12 hours, about 18 hours, about 24 hours, or about 36 hours, to about 30 minutes, about 1 hour, about 2 hours, about 3 hours, about 4 hours, about 5 hours, about 6 hours, about 9 hours, about 12 hours, about 18 hours, about 24 hours, about 36 hours, or about 48 hours.

In some embodiments, the inhibitor is administered from about 30 minutes to about 24 hours before the cancer vaccine is administered. In some embodiments, the inhibitor is administered from about 2 hours to about 4 hours before the cancer vaccine is administered. In some embodiments, the inhibitor is administered about 2 hours before the cancer vaccine is administered.

In some embodiments, the interferon comprises type 1 interferon (T1IFN) and/or type 2 interferon (T2IFN). The T1IFNs consist of 14 different -α isoforms (subtypes with slightly different specificities), and single -β, -ε, -κ, -ω, and -ζ isoforms. In some embodiments, IFN signaling inhibition targets T1IFN and in others it targets T2IFN. In still other embodiments, both T1IFN and T2IFN are inhibited. In some embodiments, the IFN signaling inhibitor comprises an antibody or fragment thereof that binds to and blocks signaling through the T1IFN (IFNAR1 and IFNAR2) and/or T2IFN (IFNGR1 and IFNGR2) receptors or that directly binds T2IFN and/or the various isoforms of T1IFN to neutralize their signaling.

Antibodies to T1IFN and T2IFN as well as antibodies to IFNAR are known and commercially available. In some embodiments, the antibody is monoclonal. In some embodiments, the antibody binds to a mouse antigen. In some embodiments, the antibody binds to a human antigen. In some embodiments, the antibody is engineered to include a human fragment crystallizable (Fc) region.

In some embodiments, the inhibitor comprises an antibody, or fragment thereof, that specifically binds and neutralizes IFNα and/or IFN3. In some embodiments, the antibody comprises anti-IFNα, such as clone TIF-3C5 (Leinco Prod #T701), clone EBI-1 (Thermo Fisher Scientific Cat #BMS160), or clone 7N41 (Thermo Fisher Scientific Cat #M710). In some embodiments, the antibody comprises anti-IFNβ, such as clone HDβ-4A7 (Leinco Prod #B659), A1 (IFNb) (Thermo Fisher Scientific Cat #16-9978-81), or MMHB-3 (Thermo Fisher Scientific Cat #214001).

In some embodiments, the inhibitor comprises an antibody, or fragment thereof, that specifically binds and blocks a T1IFN receptor. In some embodiments, the antibody comprises anti-IFNAR, such as clone MAR1-5A3 (Thermo Fisher Scientific Cat #16-5945-85), MEDI546(Anifrolmab) (Creative Biolabs Cat #TAB-722), H3K1 (Creative Biolabs Cat #HPAB-0203CQ), H2K1 (Creative Biolabs Cat #HPAB-J0127-YC) and ch64G12 (Creative Biolabs Cat #HPAB-J0126-YC).

In some embodiments, the inhibitor is an antibody, or fragment thereof, that binds and neutralizes IFNγ. In some embodiments, the antibody comprises anti-IFNγ, such as clone XMG1. 2 (Thermo Fisher Scientific Cat #12-7311-82), NIB42 (Thermo Fisher Scientific Cat #16-7318-81) or MD-1 (Thermo Fisher Scientific Cat #16-7317-85).

In some embodiments, the inhibitor is an antibody, or fragment thereof, that specifically binds and blocks a T2IFN receptor. In some embodiments, the antibody comprises anti-IFNGR, such as clone GR-20 (Thermo Fisher Scientific Cat #16-1193-85) or GIR-208 (Leinco Prod #G737).

In further embodiments, IFN signaling inhibition may encompass a small molecule inhibitor or a recombinant protein with known function to inhibit signaling through the IFN pathways. In some embodiments, the small molecule inhibitor is (3,4-dichloro-5-phenyl-5H-furan-2-one), dimethyl fumarate or a compound described in Thoidingjam et al. (2022), incorporated herein by reference²⁹.

In some embodiments, the inhibitor may comprise a recombinant protein, or a binding nucleic acid, such as an aptamer.

In some embodiments, the cancer vaccine comprises an oncolytic virus that expresses a tumor antigen, which can also be referred to as a vaccination vector, such as an oncolytic virus vector with engineered expression of a tumor antigen. The vaccine may also be derived from an alternate vaccine platform, non-limiting examples of which include non-oncolytic viral vectors, DNA vaccines, mRNA vaccines, peptide vaccines or dendritic cell vaccines.

In some embodiments, the oncolytic virus is selected from the group consisting of Herpesviridae, Rhabdoviridae, Picornaviradae, Reoviridae, Togaviridae, Adenoviridae, Paramyxoviridae, and Poxviridae.

In some embodiments, the oncolytic virus is a rhabdovirus. In some embodiments, the rhabdovirus is a vesiculovirus. In some embodiments, the rhabdovirus is a recombinant or wildtype vesicular stomatitis virus (VSV). In some embodiments, the VSV is VSVΔM51, which is an oncolytic attenuated variant of the VSV Indiana strain.

In some embodiments, the cancer vaccine and/or the inhibitor is administered by injection. In some embodiments, injection is intravascular, intratumoral, intraperitoneal, intramuscular, intradermal and/or subcutaneous. In some embodiments, injection is intravascular, including intravenous.

As used herein, the phrase “effective amount” or “therapeutically effective amount” means an amount effective, at dosages and for periods of time necessary to achieve the desired result. For example, in the context of treating cancer, an effective amount is an amount that, for example, reduces the size and/or growth of a tumor compared to the response obtained without administration of the inhibitor and/or cancer vaccine disclosed herein. Effective amounts may vary according to factors such as the disease state, age, sex and weight of the subject. The amount of a given compound that will correspond to such an amount will vary depending upon various factors, such as the given drug or compound, the pharmaceutical formulation, the route of administration, the administration schedule, the identity of the patient being treated, and the like, but can nevertheless be routinely determined by one skilled in the art.

In some embodiments, the tumor antigen is a tumor-associated antigen (TAA) of the cancer. In some embodiments, the TAA is alphafetoprotein (AFP), carcinoembryonic antigen (CEA), CA 125, Her2, dopachrome tautomerase (DCT), GP100, Melan-A/MART-1, MAGE proteins, BAGE proteins, GAGE proteins, NY-ESO1, WT-1, survivin, tyrosinase, SSX2, Cyclin-A1, KIF20A, MUC5AC, Meloe, Lengsin, Kallikrein 4, IGF2B3, glypican-3, HPV E6 and HPV E7. In some embodiments, the TAA is DCT.

In some embodiments, the tumor antigen is a tumor-specific antigen (TSA) of the cancer. In some embodiments, the TSA is referred to as a neoantigen. In some embodiments, the TSA is an abnormal product of ras or p53 gene.

In some embodiments, the method further comprises administering adoptive cell transfer (ACT) cells and/or a checkpoint inhibitor. In related embodiments, tumor-specific vaccination and IFN inhibition are further combined with adoptive T cell therapy (ACT). In related embodiments, tumor-specific vaccination and IFN inhibition are further combined with checkpoint inhibitor blockade (CIB).

In some embodiments, the ACT cells are derived from tumor infiltrating lymphocytes (TILs) or peripheral blood mononuclear cells (PBMCs) having a histocompatible phenotype to the subject. In some embodiments, the ACT cells are autologous PBMCs. In some embodiments, the ACT cells are transduced with a recombinant tumor antigen-specific receptor, a T cell receptor (TCR) or a chimeric antigen receptor (CAR). In some embodiments, the ACT cells comprise CD8 T cells. In some embodiments, the CD8 T cells are stem cell memory, central memory, effector memory or effector phenotype T cells.

In some embodiments, the checkpoint inhibitor inhibits checkpoint proteins. In some embodiments, the checkpoint proteins comprise CTLA-4, PD-1, PD-L1, PD-L2, LAG3, TIGIT and/or TIM3.

In some embodiments, the inhibitor of interferon (IFN) signaling and/or the cancer vaccine comprising a tumor antigen may be formulated as a composition with at least one pharmaceutically acceptable carrier, diluent or excipient. As used herein, a “pharmaceutically acceptable carrier, diluent, or excipient” includes any material which, when combined with an active ingredient, allows the ingredient to retain biological activity and is non-reactive with the subject's immune system. Examples include, but are not limited to, any of the standard pharmaceutical carriers such as a phosphate buffered saline solution, water, emulsions such as oil/water emulsion, and various types of wetting agents. In some embodiments, diluents for aerosol or parenteral administration are phosphate buffered saline (PBS) or normal (0.9%) saline. Compositions comprising such carriers are formulated by well-known conventional methods (see, for example, Remington's Pharmaceutical Sciences, 18th edition, A. Gennaro, ed., Mack Publishing Co., Easton, Pa., 1990; and Remington, The Science and Practice of Pharmacy 20th Ed. Mack Publishing, 2000). On this basis, the composition includes, albeit not exclusively, buffered solutions with a suitable pH and iso-osmotic with physiological fluids.

In another aspect of the present disclosure, provided is a kit comprising an inhibitor of interferon (IFN) signaling and optionally a pharmaceutically acceptable carrier, diluent or excipient, a cancer vaccine comprising a tumor antigen and optionally a pharmaceutically acceptable carrier, diluent or excipient, and instructions for use of the kit. In some embodiments, the kit is for stimulating tumor antigen-specific immune responses. In some embodiments, the kit is for treating cancer. In some embodiments, the kit also includes a container, optionally having a sterile access port (for example the container may be an intravenous solution bag or a vial having a stopper pierceable by a hypodermic injection needle) and/or applicator, e.g., single and multi-chambered pre-filled syringes (e.g., liquid syringes and lyosyringes).

### EXAMPLES

The following non-limiting examples are illustrative of the present disclosure. Specific elements of the examples are for descriptive purposes only and are not intended to limit the scope of the invention. Those skilled in the art could develop equivalent methods and utilize comparable materials that are within the scope of the invention.

### Experimental Methods and Procedures

Mice

All mice were bred and/or housed in the Central Animal Facility at McMaster University, a specific pathogen-free facility. Thy1.1 mice (B6.PL-Thy1a/CyJ) and BATF3^{-/-} mice (B6.129S(C)-Batf3^{tm1Kmm/J}) were purchased from The Jackson Laboratory. P14 mice (B6.Cg-TcratmlMom Tg(TcrLCMV)327Sdz), a transgenic mouse strain that carries a T cell receptor (TCR) recognizing an H-2db-restricted epitope of the lymphocytic choriomeningitis virus (LCMV) glycoprotein 33 (gp33)—LCMV-GP33-41 (KAVYNFATM (SEQ ID NO:1))—were purchased from Taconic Breeding Laboratories and were cross-bred to Thy1.1 mice to generate P14Thy1.1 mice. C57Bl/6 mice were purchased from Charles River Laboratories.

Cell Lines and Tumor Challenge

All cells were maintained at 37° C. in a humidified atmosphere with 5% CO₂, MO5, B16F10 and B16gp33 cells (B16F10 cells stably transfected with a minigene corresponding to the gp33 peptide)³⁰ were maintained in MEM/F11 containing 10% FBS, 2 mM 1-glutamine, 5 ml sodium pyruvate, 5 mL nonessential amino acids, 5 mL vitamin solution (Thermo Fisher Scientific), 55 μM 2-mercaptoethanol (Sigma-Aldrich), 100 U/mL penicillin, and 100 ng/ml streptomycin. Vero, HEK 293T, MCA205gp33 and MC38gp33 cells were cultured in DMEM supplemented with 10% FBS, penicillin/streptomycin (100 U/mL and 100 ng/mL, respectively), and 2 mM 1-glutamine (Thermo Fisher Scientific). These cells were generated via transduction of MCA205 and MC38 cells, respectively, with a lentivirus (pLV-EFlaIRESpuro was a gift from Tobias Meyer (Addgene plasmid #85132; http://n2t.net/addgene:85132; RRID: Addgene_85132))³¹ engineered to express the gp33 peptide (KAVYFATM (SEQ ID NO:1)) like described previously for other peptides in other cell lines²⁷. Tumor cells were washed twice with PBS and resuspended in PBS at a concentration of 10⁶ cells/30 μL for MCA205gp33 cells or 10⁵ cells/30 μL for B16gp33, MO5 and MC38gp33 cells. Mice were challenged via intradermal (i.d.) injection, and tumors were allowed to grow to a mean volume of approximately 150 mm³ prior to the commencement of treatment.

Tumor-primed CD8 T cells were generated via intravenous injection of naïve CD8 T cells isolated from P14 splenocytes using a CD8⁺ negative magnetic selection kit (StemCell Technologies) into naïve C57BL6 mice one day prior to B16gp33 tumor implantation. Thy1.2 depletion antibody was used one day prior to T cell injection to maximize engraftment. Thy1.1⁺CD8⁺ cells subsequently extracted from the inguinal, brachial, and axillary lymph nodes were shown to be antigen experienced and were used as tumor-primed T cells.

Antibodies

The αIFNα(clone TIF-3C5), αIFNβ(clone HDβ-4A7), αIFNGR (clone GR-20), αIFNAR (clone MAR1 5A3) and αPD-L1 (clone 10F.9G2) antibodies were purchased from Leinco or Cedarlane, isotype control antibody (clone HRPN) was purchased from Bio X cell and the αIFNγ antibody (clone XMG1. 2) was prepared and purified in house. Thy1.2 depletion antibody (clone 30H12) was purchased from Cedarlane Laboratories.

Viruses

Vesicular stomatitis virus (VSV) was propagated, purified and quantified on Vero or HEK 293T cells as known in the art³². Briefly, virus stocks were purified from cell culture supernatants by centrifugation (780×g) to sediment dead cells before filtration through a 0.22 μm Steritop filter (Millipore) and centrifugation at 30,000×g for 1.5 hours to pellet virus. Virus pellet was resuspended in PBS and loaded on top of a continuous gradient spanning to 15% to 50% Optiprep (Sigma) and centrifuged at 160,000×g for 1.5 hours. Subsequently, a single white opaque band was observed, which was collected and frozen in aliquots at −80° C. Plaque assay on Vero cells was used to titer virus stocks. VSV-gp33 is a recombinant VSV that expresses the dominant CD8+ and CD4+ T cell epitopes of the LCMV glycoprotein (LCMV-gp33-41 and LCMV-gp61-80, respectively) in a minigene cassette. VSV-DCT is a recombinant VSV that expresses the full-length human DCT³³ and VSV-SIINFEKL-Luc is the same with a modified version of luciferase (Luc) linked to the immunodominant class-I epitope from OVA (SIINFEKL (SEQ ID NO:2))³⁴. VSV-GFP is a recombinant VSV that expresses green fluorescent protein (GFP). VSV-gp33, VSV-DCT, VSV-SIINFEKL-Luc and VSV-GFP were modified to abrogate their ability to inhibit IFN-α/-β responses via deletion of the methionine residue at position 51 of the matrix protein³⁵.

Peptides

Peptides for gp33 (KAVYNFATM (SEQ ID NO:1)), OVA (SIINFEKL (SEQ ID NO:2)), DCT (SVYDFFVWL (SEQ ID NO:3)) and RGY (RGYVYQGL (SEQ ID NO:4)), were purchased from Biomer Technologies and dissolved in DMSO.

Antibody and Vaccine Treatments

Antibodies were diluted in PBS and administered by intraperitoneal (i.p.) injection between 24 and 2 hours before virus injection. A single dose of 1 mg per mouse was given of each antibody. Approximately 2-4 hours after antibody treatment, 2×10⁸ pfu of VSV was administered via tail vein injection. About 150 μL blood was collected via retro-orbital bleed at 1, 5, 7, 12 and 21 days (one or more of those timepoints as indicated in the results) following treatment for immune analysis. Tumor volumes were monitored and measured every 2-3 days until they reached their endpoint volume (1,000 mm³).

Surface and Intracellular Staining of T Cells

The following reagents and antibodies for flow cytometric analysis were purchased from BD Biosciences: Fc block (catalog 553141), 7AAD (catalog 559925), Fixable Viability Stain 510 (catalog 564406), BV786 rat anti-mouse CD3 (clone 17A2), Pacific Blue or PE-Cy7 rat anti-mouse CD8a (clone 53-6.7), PE rat anti-mouse CD4 (clone GK1.5) or APC-Cy7 rat anti-mouse CD4 (clone GK1.5), PE mouse anti-rat Thy1.1 (clone OX-7), Alexa Fluor 700 rat anti-mouse CD62L (clone MEL-14), and FITC rat anti-mouse CD44 (clone IM-7). BV711 rat anti-mouse CD274 (PD-L1; clone MIHS), BV650 rat anti-mouse CD279 (PD-1; clone RMP1-30), BV421 mouse anti-mouse CD366 (TIM-3; clone 5D12), APC rat anti-mouse IFNγ(clone XMG1.2), BV421 hamster anti-mouse CD11c (clone HL3), BV650 rat anti-mouse F4/80 (clone T45-2342), PerCP-Cy5.5 rat anti-mouse Ly6G (clone 1A8), FITC rat anti-mouse Ly6C (clone AL 21), PE-Cy7 rat anti-mouse CD19 (clone 1D3), BV605 rat anti-mouse CD11b (clone M1/70).

Briefly, staining was performed on venous blood, spleen and lymph node samples that were treated with ACK lysis buffer to remove red blood cells (RBCs) prior to peptide stimulation and/or staining. Cells were treated with Fc Block and stained for surface markers followed by viability staining. For analysis of antigen-specific responses, PBMCs were extracted from blood samples using RBC lysis buffer and stimulated with DCT, OVA, RGY or gp33 peptide (1 μg/mL) in culture at 37° C. for 4 hours. Brefeldin A (GolgiPlug, BD Biosciences; 1 μg/mL) was added for the last 3 hours of incubation. Blocking and surface staining were performed as above except that the cells were stained with fixable viability dye before fixation/permeabilization (Cytofix/Cytoperm, BD Biosciences) and intracellular staining for IFNγ and TNFα expression. For the antigen presentation assay, CD8 T cells were purified from P14 splenocytes using an EasySep™ Mouse CD8+ T Cell Isolation Kit (STEMCELL Technologies) and labeled with CellTrace™ Violet proliferation kit (ThermoFisher Scientific) according to manufacturer's instructions. Cells (1×10⁶ cells/mouse) were transferred into a B16gp33 tumor bearing mouse via IV injection at varying time points after treatment. Tissues were harvested 3 days later, and immune cells extracted before staining for Thy1.1 and CD8. The proportion of Thy1.1⁺CD8⁺ T cells that had divided at least once were then used for subsequent antigen presentation analysis. Overall, fluorescence was detected using either a BD LSRFortessa or LSR II flow cytometer (BD Biosciences). Proliferation dye dilution analysis was performed using FCS Express 7 and all other flow cytometry data were analyzed using FlowJo (version 10) flow cytometry analysis software (Tree Star).

Immunohistochemistry

Tissue staining with CD8 and VSV antibodies was performed on sections from formalin-fixed, paraffin-embedded tumor tissues using a Leica Bond Rx automated stainer (Leica Biosystem). For CD8 staining, slides were dewaxed and pretreated with Leica Bond Epitope Retrieval buffer #2 (Leica Biosystems) for 20 minutes before staining with rat anti-mouse CD8a antibody (diluted 1:1000; clone 4SM15, Thermo Fisher Scientific). Color was developed using the Leica Bond Polymer Refine Detection Kit (Leica Biosystems), substituting the post primary component with rabbit anti-rat antibody (1:100, Vector Laboratories). For VSV staining, Slides were dewaxed and pretreated with Leica Bond Epitope Retrieval buffer #1 (Leica Biosystems) for 20 minutes before staining with rabbit VSV antiserum (diluted 1:5000; Imanis Life Sciences). Color was developed using the BOND Polymer Refine Red Detection kit (Leica Biosystems), using a protocol that omits the Post Primary reagent and only uses the anti-rabbit polymer. Images were taken using a Zeiss Imager M2 Microscope (Zeiss).

For PD-L1 immunohistochemistry, tumors were harvested, and flash frozen in OCT. Frozen tissue sections were cut at 5 μm onto coated slides. Sections were air dried overnight and then fixed in 10% Formalin for 5 min before being treated with 1% H₂O₂ in dH₂O for 15 min at room temperature. Slides were then wash in dH₂O to remove excess H₂O₂. Slides were rinsed in Bond Wash (Leica) and placed on the Leica Bond Automated stainer. The slides were stained with Rat primary PD-L1 (Clone M1H5, Thermo Fisher Scientific) diluted 1:500 in Animal Free Diluent (Vector Labs SP-5035). The BOND Polymer Refine Red Detection kit (Leica) was used according to the manufacturer's protocol. Images were taken using the Aperio Slide Scanner and analyzed using ImageScope v11.1.2.760 software (Aperio).

Statistics

GraphPad Prism for Windows was used for graphing and statistical analyses. Mean+SD bars are shown. Differences between means of immune response data were queried using either paired Student's 2-tailed T test where a single time point was assessed or a 2-way ANOVA with mixed-effects model. Where necessary, the Holm-Sidik method was used to correct for multiple comparisons. Survival curves were generated using the Kaplan-Meier method, with a tumor volume of 1000 mm³ or tumor ulceration as end point and analyzed using the log-rank (Mantel-Cox) test. *P<0.05; **P<0.01; ***P<0.001; ****P<0.0001.

Study Approval

All animal experiments were compliant with Canadian Council on Animal Care guidelines and received internal approval through the McMaster University Animal Research Ethics Board.

### Results

Type I IFN Differentially Regulates Vaccine-Induced Proliferation of Endogenous and Adoptively Transferred T Cells

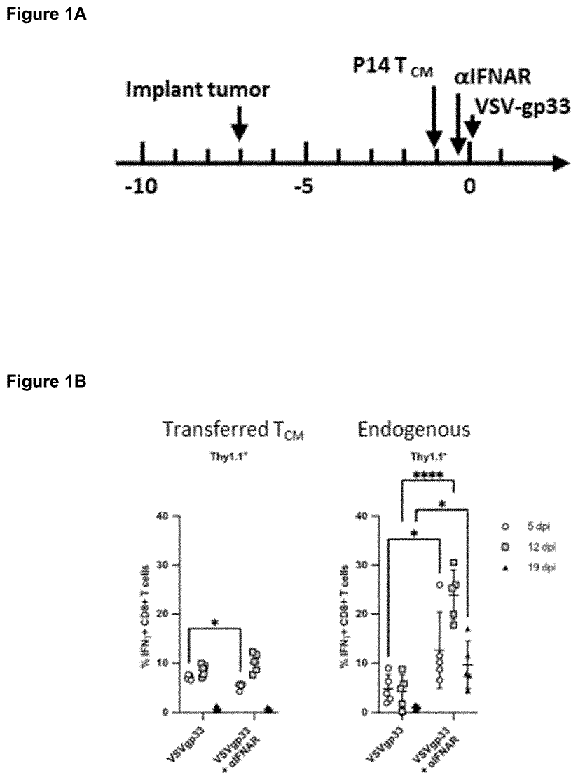

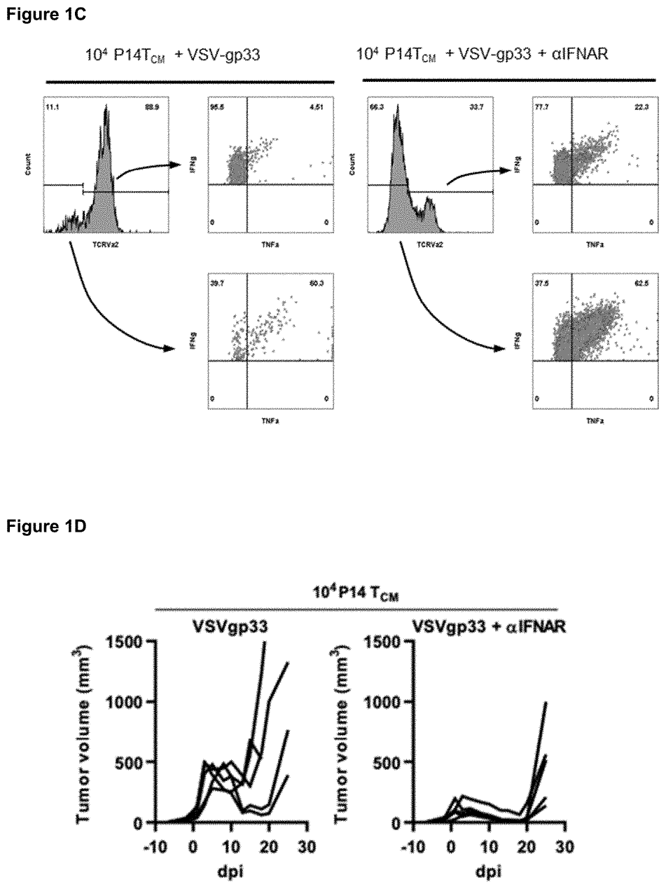

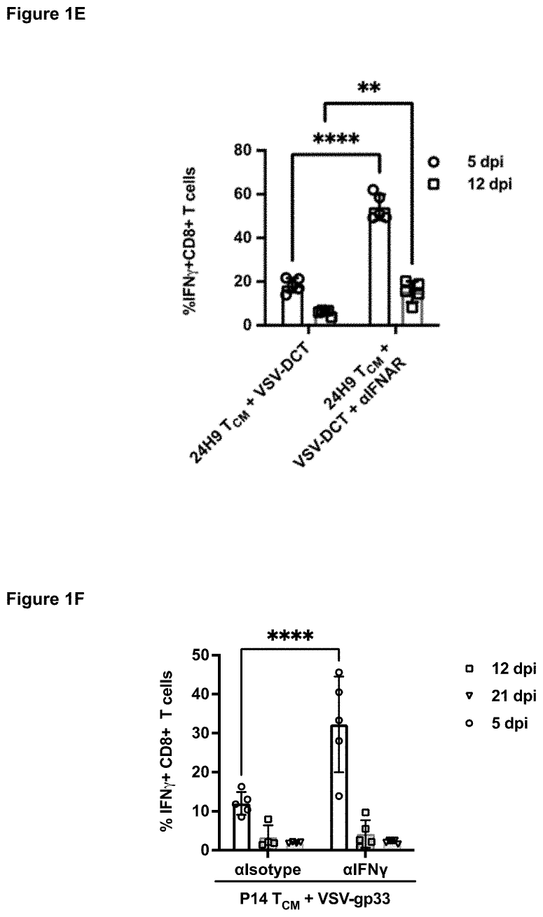

The influence of transient IFNAR blockade on cancer immunotherapy and anti-tumor immunity was first evaluated. An established B16gp33 tumor model (B16F10 melanoma cell line expressing gp33, a lymphocytic choriomeningitis virus glycoprotein-derived peptide) treated with a previously established combination therapy of adoptive transfer of central memory T cells (T_{CM} ACT) and oncolytic virus vaccination (OVV) was used in the presence or absence of anti-IFNAR antibody (αIFNAR) (FIG. 1A)²⁸. Specifically, B16gp33-bearing C57Bl/6 mice were infused with in vitro differentiated P14 memory T cells (Thy1.1V) that recognize the gp33 epitope, followed by vaccination with vesicular stomatitis virus (VSV) expressing gp33 (VSV-gp33). The dosing of IFNAR antibody was modified from previous studies to a more transient effect by using a single dose of 1 mg just prior to virus treatment (approximately 2-4 hours) in order to focus the effect of IFNAR blockade on the stimulation of anti-tumor responses. In addition, the sensitivity of the model was increased by transferring a lower number of P14 cells (10⁴) than was used previously.

Antigen-specific T cell responses were numerated by peptide stimulated IFNγproduction at different time points. As shown in FIG. 1B, gp33-specific T cell numbers peaked on day 5 and were sustained for at least 19 days when IFNAR antibody was part of the treatment whereas T cell responses progressively decreased in the absence of IFNAR antibody. IFNAR antibody treatment did not change the magnitude of responses derived from transferred P14 T cells (Thy1.1⁺ in FIG. 1B), suggesting that transferred T cell proliferation is not influenced by T1IFN signaling. Surprisingly, IFNAR blockade dramatically increased endogenous T cell responses against gp33 (Thy1.1⁻ in FIG. 1). In addition, a higher proportion of endogenous cells were double positive for IFNγ and TNFα compared to transferred cells (FIG. 1C), a characteristic that has been previously correlated with increased functionality and cytotoxicity^{36,37}. Double positive gp33-reactive cells were observed in mice treated with virus alone, but this effect was proportionally higher and more striking in those pretreated with IFNAR antibody due to the increased number of antigen-specific endogenous T cells. Anti-tumor effect was observed in both groups but was less consistent and thorough in the mice receiving virus alone compared to those that also received IFNAR antibody treatment. Regression began concurrent with the peak of T cell response at 5 days post infection (dpi) and approaching complete regression between 10 and 20 dpi in the IFNAR blockade group before relapsing thereafter (FIG. 1D), despite the persistence of strong gp33 responses past 19 dpi (FIG. 1). Relapse in this model was previously characterized and it was found that emergence of variant cells lacking the gp33 transgene is common, which is likely also the case here and explains why gp33 responses were unable to control relapse tumor growth³⁸. Furthermore, a similar increase in antigen-specific responses was seen when using IFNAR blockade in combination with VSV and dopachrome tautomerase (DCT; a melanoma differentiation antigen otherwise known as TRP-2) vaccination (VSV-DCT), and 24H9 T_{CM} ACT, also within the B16gp33 model (FIG. 1E). Improvement in the magnitude of T cell responses was also observed when IFNγsignaling was neutralized, suggesting a role of common targets of T1IFN and T2IFN signaling (FIG. 1F).

Taken together these results suggest an impairment of endogenous antigen-specific T cell responses, which is not shared by transferred cells but can be rescued by IFNAR blockade. This has an important implication in the rational design of cancer vaccines as they are often formulated to maximize T1IFN induction and signaling in T cells as it is thought to benefit therapeutic outcomes. However, the results suggest that minimizing or blocking T1IFN signaling in endogenous tumor-primed T cells generates maximal therapeutic responses and outcomes from cancer vaccines.

IFN Blockade Enhances CD8+ T Cell Response to Cancer Vaccination

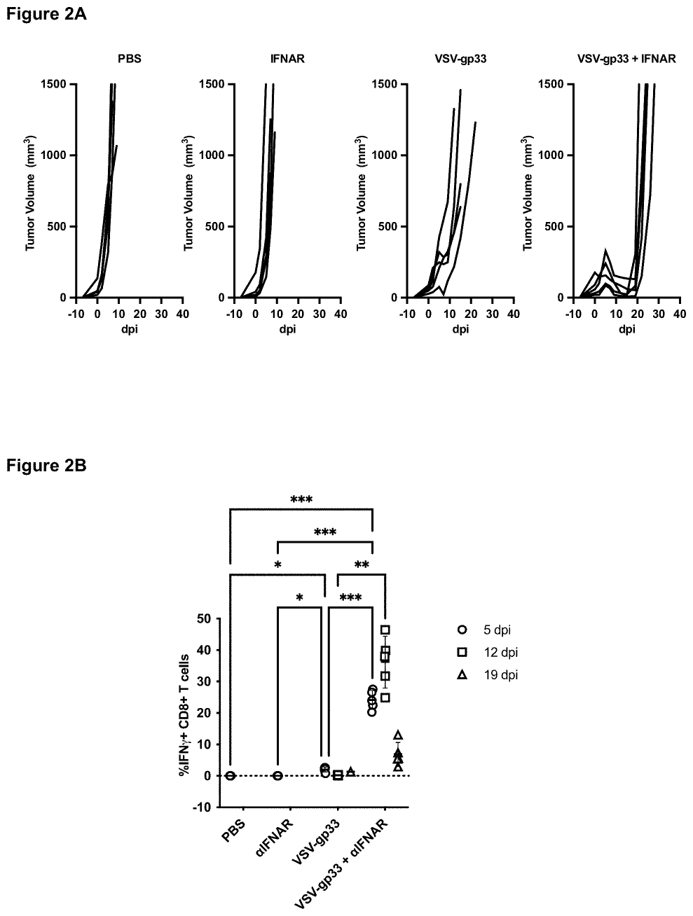

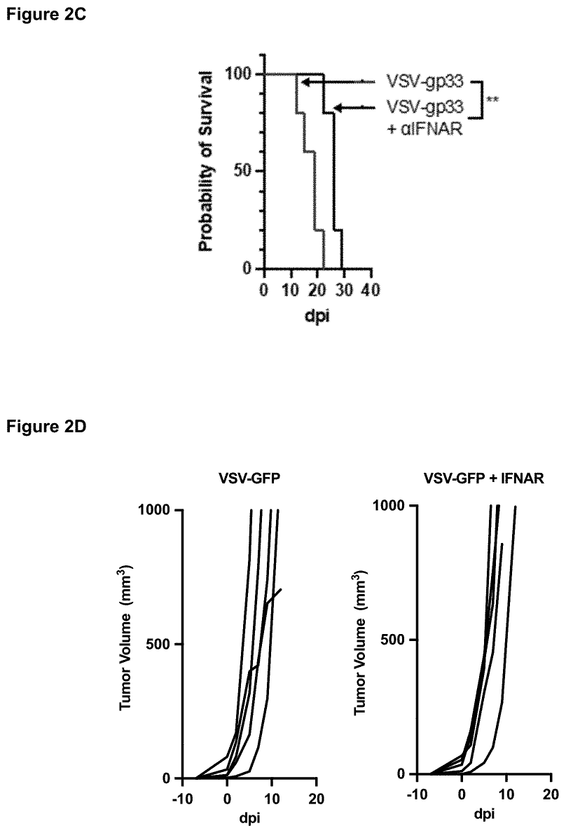

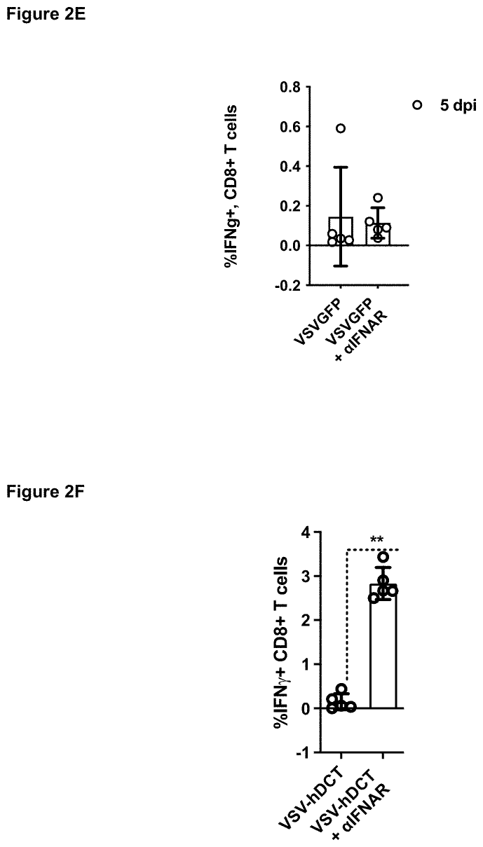

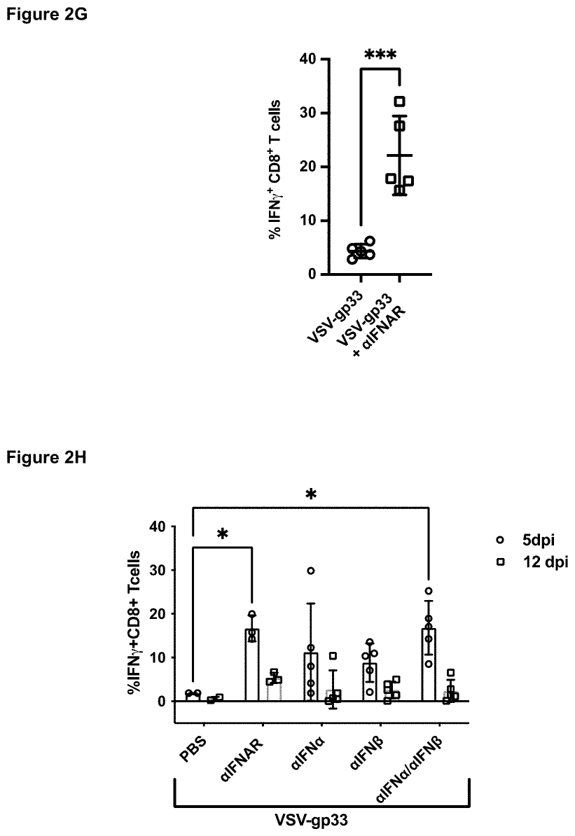

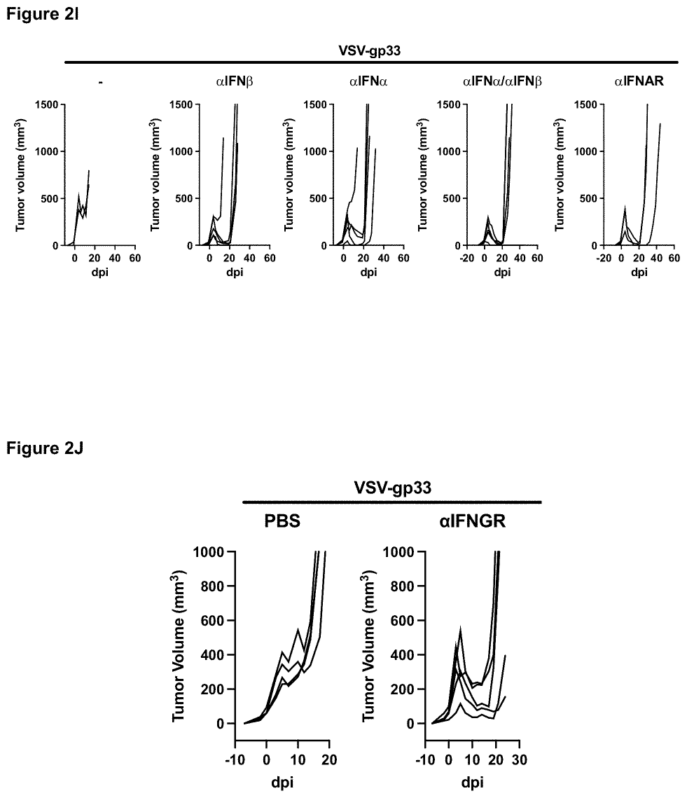

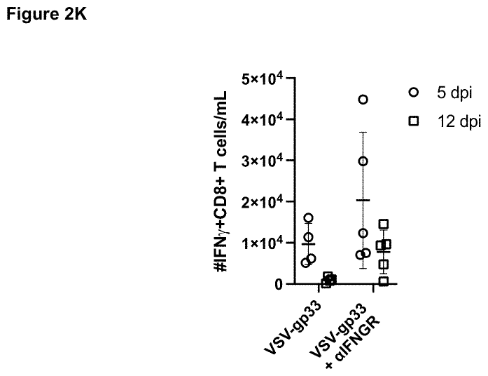

To more precisely evaluate the impact of IFNAR blockade on the response of endogenous T cells to vaccination, the B16gp33 tumor model was once again used with VSV-gp33 vaccination, but in the absence of transferred P14 T cells. As before, an equivalent volume of PBS was given as a treatment control, showing only marginal but detectable responses against gp33 at five dpi but no control of tumor growth (FIG. 2A and FIG. 2B). Tumor control and response quantification were similar in IFNAR blockade alone treated mice with no discernable effect compared to PBS controls (FIG. 2A and FIG. 2B). VSV-gp33 treatment induced a small gp33-specific response, peaking at five dpi with responses ranging from one to three percent (FIG. 2B). A minor effect was observed on tumor growth with kinetics correlating closely with gp33-specific responses, causing a temporary plateau or regression between three and 10 dpi and somewhat prolonged survival, although not significantly (FIG. 2A). However, responses were short lived, diminishing to a nominal level by 12 dpi and failing to completely regress the tumors before a robust outgrowth of the tumor (FIG. 2A and FIG. 2B). Combination of IFNAR blockade with VSV-gp33 vaccination produced a significant increase in the level of gp33-specific T cells response compared to VSV-gp33 alone (>10 fold increase in FIG. 2B) with a proportional enhanced effect on tumor growth resulting in complete or near complete regression and significantly prolonged survival before antigen negative relapse (FIG. 2B and FIG. 2C). IFNAR blockade had no effect on tumor growth or gp33-specific T cell responses when combined with a VSV lacking antigen expression (VSV-GFP in FIG. 2D and FIG. 2E). These results suggest that any effect on oncolysis (otherwise known as tumor cell killing derived directly from virus infection/replication) was negligible and that vaccination with a vector encoding a tumor antigen was required for clinical benefit from IFNAR blockade.

To extend this observation to a different peptide target in the same tumor model, tumor bearing mice were vaccinated against DCT. Here, a xenoimmunization strategy was employed using a VSV vector engineered to express the human DCT as done previously³⁹. The immunodominant epitope is completely conserved between murine and human DCT protein and so this vaccination strategy is robust. Again, DCT-specific responses were augmented by IFNAR blockade treatment used in combination with VSV-hDCT compared to the virus alone treated mice (FIG. 2F), demonstrating that the effect of IFNAR blockade is not limited to the gp33 peptide target. Furthermore, a similar pattern is also seen when the virus was given by an alternate route, with intratumorally injected mice showing an increased gp33-specific response in IFNAR blockade treated mice compared to virus alone treatment (FIG. 2G).

With IFNAR blockade showing such dramatic results, inhibition of T1IFN signaling by other means was also assessed. Thus, the treatment of B16gp33 tumor bearing mice with VSV-gp33 was repeated but included groups receiving antibodies with neutralization activity against IFNα or IFNβ. Treatment with αIFNα or αIFNβ separately showed an improvement in magnitude of gp33-specific T cell response and tumor regression compared to virus alone controls but they failed to reach similar levels to IFNAR blockade (FIG. 2H and FIG. 2I). However, the combination of both neutralizing antibodies did reach similar levels, indicating that blocking of all T1IFN is required for optimal effect (FIG. 2H and FIG. 2I). Although the IFNα neutralizing antibody shows function against the majority of IFNα subtypes, its neutralization is incomplete against select subtypes. Therefore, it is not surprising that the neutralizing antibody treated groups showed more variability compared to IFNAR blockade, which blocks signaling from all subtypes of T1IFN (FIG. 2H and FIG. 2I).

The effect of blocking the T2IFN receptor from stimulation with IFNγ, which induces a signaling pathway with similar anti-proliferative effects to T1IFN, was also assessed. An antibody (denoted as αIFNGR) with similar blocking effects against the T2IFN receptor (interferon gamma receptor (IFNGR) composed of IFNGR1 and IFNGR2 heterodimer) was given to B16gp33 tumor bearing mice two hours before VSV-gp33 vaccination. Tumor regression (FIG. 2J) and gp33-specific T cell response magnitude (FIG. 2K) were increased with IFNGR blockade compared to VSV-gp33 vaccination alone, although significance was not reached. These results suggest a similar augmenting effect of IFNGR blockade compared to IFNAR blockade in the context of tumor antigen-specific vaccination.

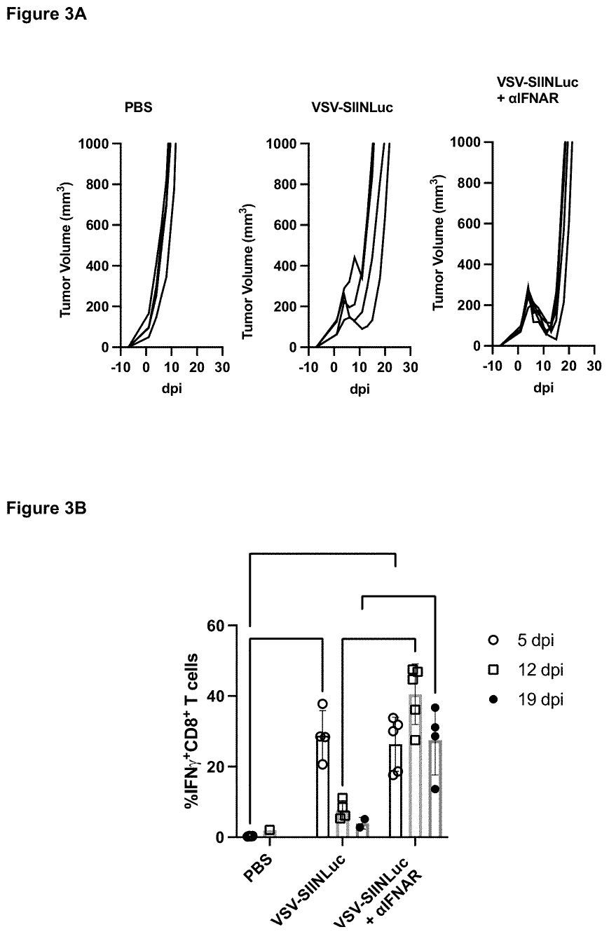

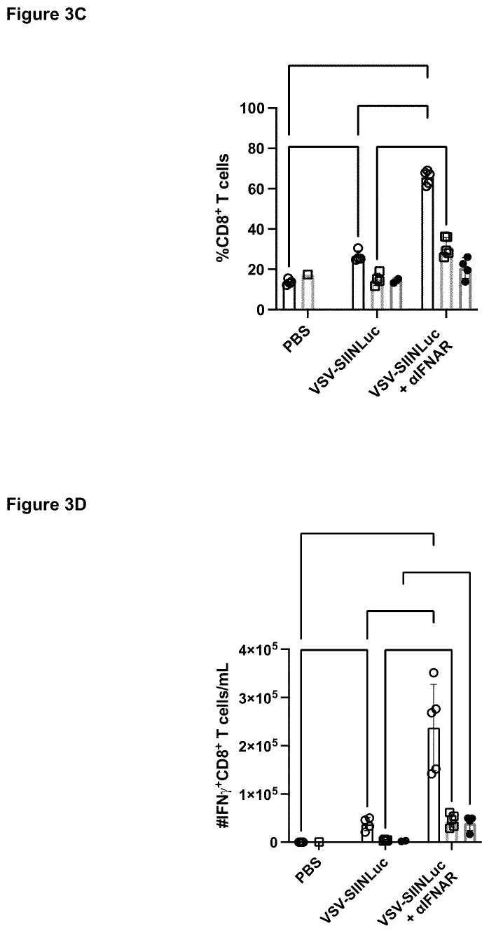

IFNAR blockade on MO5 (B16F10 cells engineered to express ovalbumin (OVA) protein) tumor-bearing mice in combination with VSV expressing SIINFEKL (SEQ ID NO:2; VSV-SIINFEKL-Luc), an OVA-derived immunodominant epitope, was then tested. Although tumor regression was enhanced by IFNAR blockade in these mice (FIG. 3A), the magnitude of T cell response was not significantly increased at the earliest time point (5 dpi in FIG. 3B). However, responses at later time points (12 and 19 dpi in FIG. 3B) were significantly elevated in IFNAR blockade treated mice compared to virus alone. However, a significant increase in the relative level of total CD8 T cell compartment was also observed (FIG. 3C) and, reasoning that this may skew the relative response magnitude when expressed as a percent of total CD8 T cells, the analysis was modified. When quantified T cell responses were expressed as total counts, more than a seven-fold increase in OVA-specific T cell response was seen when IFNAR antibody co-delivered with VSV-SIINFEKL-Luc, thereby confirming that IFN blockade-enhanced T cell responses are not limited to gp33 and that this phenomenon is not restricted to B16gp33 tumors (FIG. 3D).

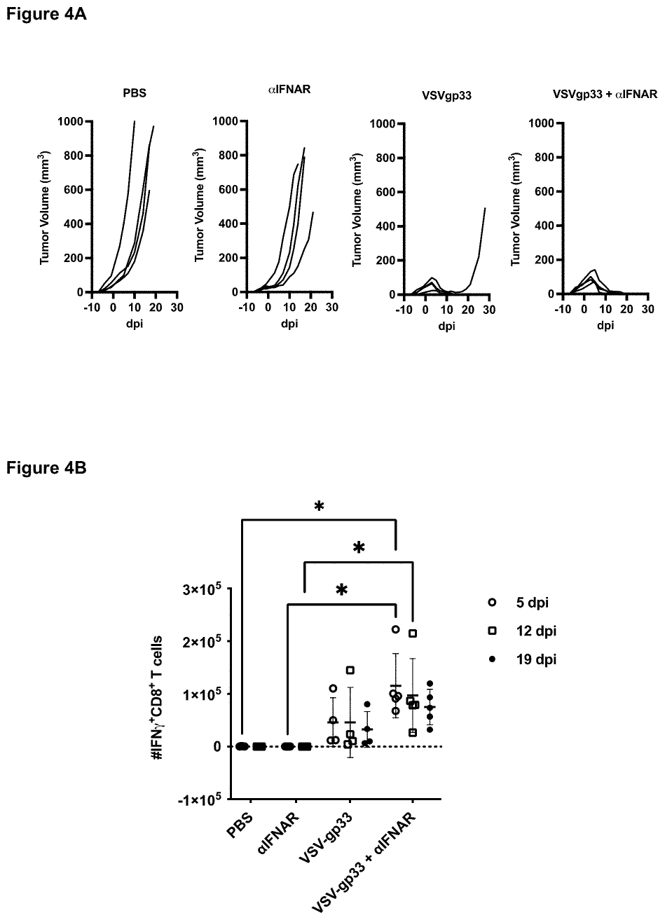

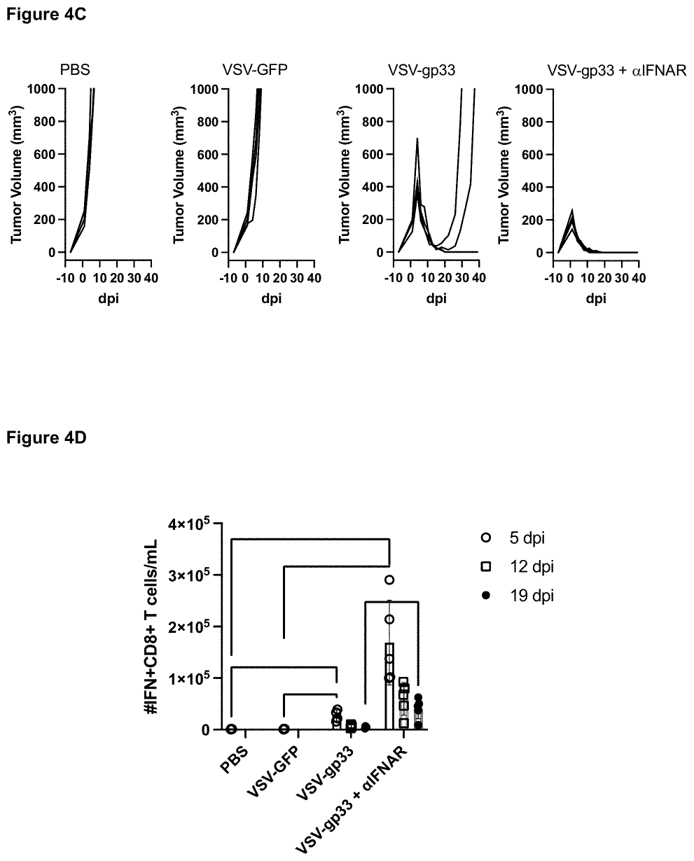

Enhancement of VSV vaccine induced antigen-specific T cell responses and anti-tumor effect by co-treatment with IFNAR blockade in several other tumor models was also observed. But, the degree of benefit and the incidence of relapse was inversely correlated with immunogenicity of the model. In the MC38 colon carcinoma cell line model, a more immunogenic model known to react robustly to other immunotherapies like CIB, virus alone treatment induced a robust response to tumor-expressed antigens, leaving less scope for IFNAR co-treatment to show an enhancement. Nonetheless, an enhancement in tumor regression and response magnitude was observed when VSV vaccination was combined with IFNAR blockade compared to vaccination alone (FIG. 4A and FIG. 4B). Finally, cured mice resisted subsequent challenge with an antigen-negative version of the initial tumor, indicating that IFNAR blockade does not have a detrimental effect on the long-term surveillance program established by tumor-specific antigen-spreaded responses. MCA205gp33 tumors, a fibrosarcoma cell line model, which have shown an intermediate immunogenicity in other studies, showed an intermediate response after VSV-gp33 alone treatment with complete tumor regression and suffering relapse in only half of the treated mice (FIG. 4C). Yet again, combination with IFNAR blockade drove enhancement in both metrics, increasing the mean magnitude of response 6-fold compared to virus alone and completely regressing all treated tumors with no relapse (FIG. 4D). Cured mice were also resistant to subsequent challenge with gp33-antigen negative parental MCA205 cells, further supporting the notion that antigen-spreaded responses were not inhibited by αIFNAR treatment.

Tumor-Primed CD8+ T Cells are the Target of IFNAR Blockade Benefit

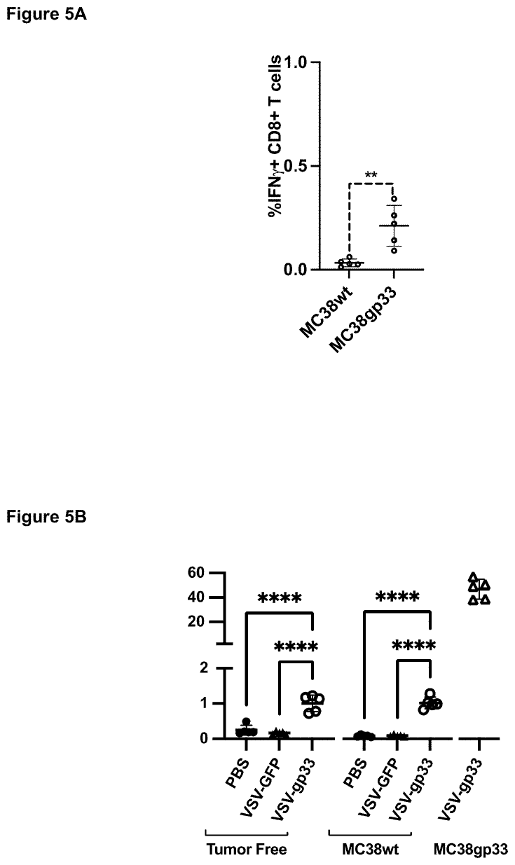

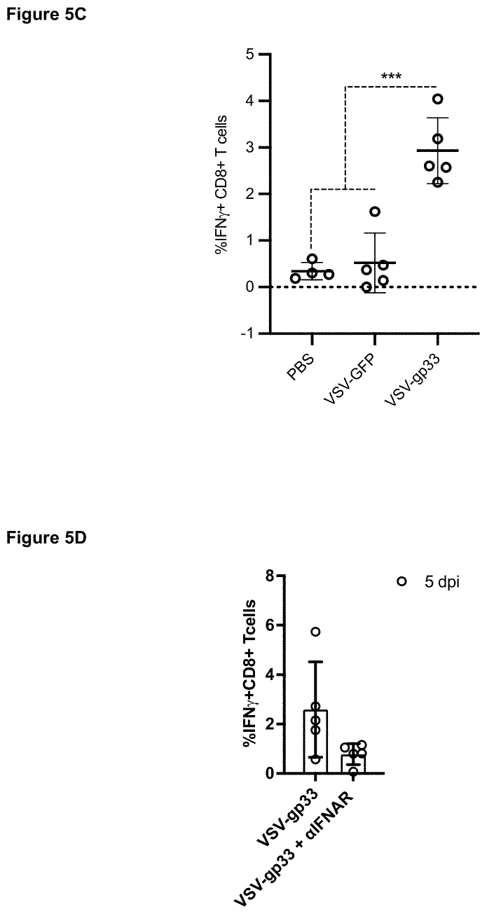

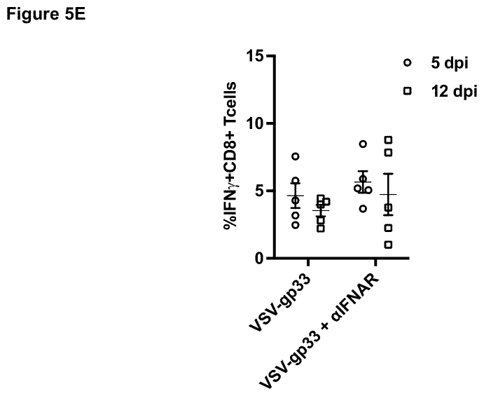

It has been previously noted that VSV excels at boosting pre-existing T cells responses but is a poor stimulator of primary responses³⁹. Thus, the large responses observed in the MC38gp33 and MCA205gp33 models may represent a boosting of pre-existing antigen experienced T cells. Indeed, gp33-specific T cells could be detected in the circulation of mice seven days after inoculation with MC38gp33 tumor cells, a time point correlative with VSV treatment, but these cells were absent in MC38 wt tumor bearing mice (FIG. 5A). Next, VSV-gp33 induced responses were compared in tumor-free mice to that of mice bearing MC38 wt or MC38gp33 tumors. Both tumor free and MC38 wt tumor bearing mice responded equivalently to vaccination, yielding an average gp33-specific response magnitude of ˜1%, which is drastically less than that observed in similarly treated MC38gp33 tumor bearing mice (FIG. 5B). Furthermore, MC38gp33 tumor bearing BATF3^{-/-} mice, which show a deficit in development of professional cross-presenting CD8⁺ and CD103⁺ DCs, were unable to generate responses of comparable levels to similarly vaccinated wildtype (wt) mice (FIG. 5C compared to FIG. 5B). Thus, VSV response magnitude appears to be dependent on expression and cross-presentation of gp33 from the growing tumor, indicating that T cells primed during tumor growth serve as a pool of pre-existing antigen-specific T cells exquisitely sensitive to VSV vaccination.

The previous data suggest that T cell responses to VSV vaccination are dominated by tumor-primed T cells. Thus, how this relates to the augmented response observed with IFNAR blockade was assessed. Data shown in FIG. 1 demonstrates that enhanced responses were derived from endogenous T cells rather than from the transferred cells, which suggests a defect in endogenous T cells derived from tumor-priming but corrected with IFNAR blockade. So, a similar benefit from IFNAR blockade was next tested with other endogenous T cell subsets. Tumor free mice similarly treated with VSV-gp33 and IFNAR blockade showed a decrease, although not a significant difference, in antigen-specific response magnitude compared to VSV-gp33 alone at early time points after treatment (FIG. 5D). Since tumor mediated expression of gp33 is absent in these mice, these responses should represent a primary response so these results correspond well with previous studies showing a negative effect of T1IFN inhibition on primary T cell responses. This notion is further supported by the fact that CD8⁺ T cell responses against RGY, an immunodominant epitope derived from VSV nucleocapsid protein, were unaffected by addition of IFNAR antibody treatment with VSV-gp33 at early time points (FIG. 5E). Thus, tumor priming appears to be essential for the augmented responses observed when IFNAR blockade is combined with VSV vaccination, with primary and more conventional memory T cells (in the form of the transferred T_{CM} cells) showing a decrease or no effect in response magnitude, respectively.

IFNAR Blockade Enhances VSV Replication and Replication-Associated Antigen Presentation

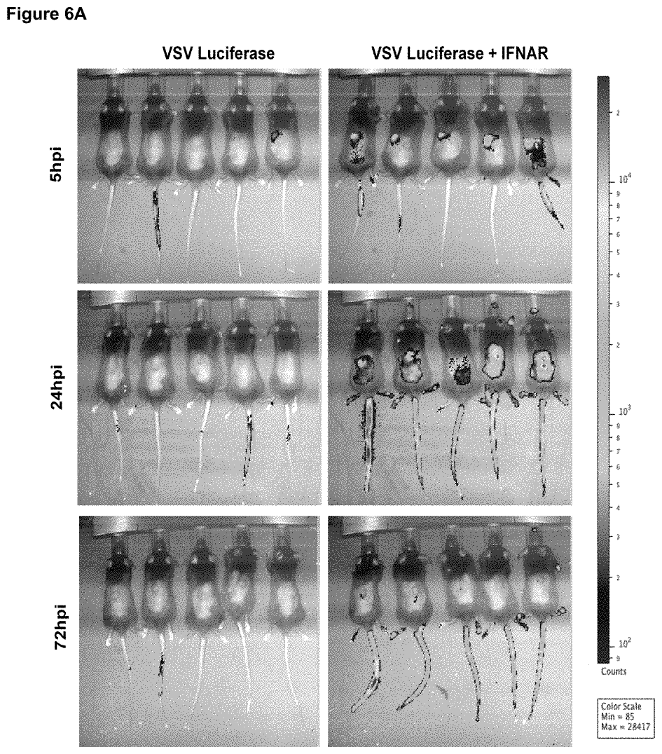

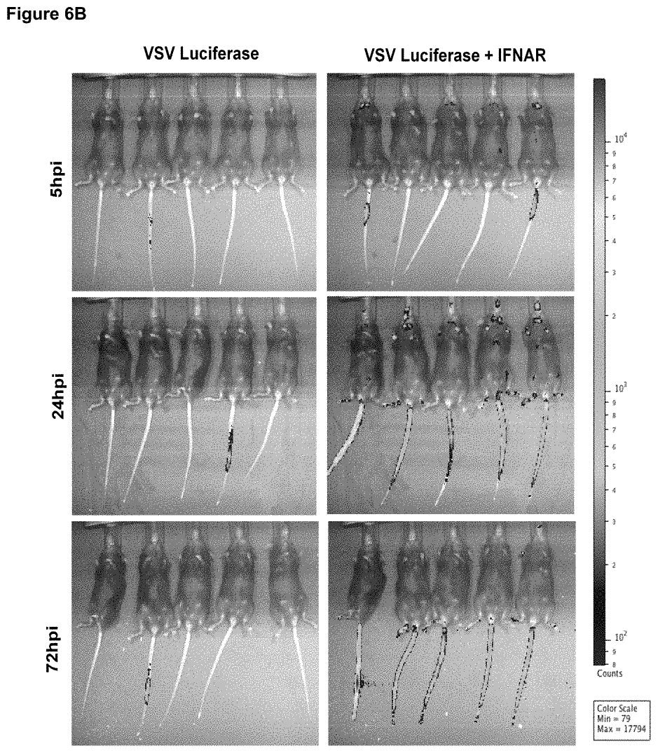

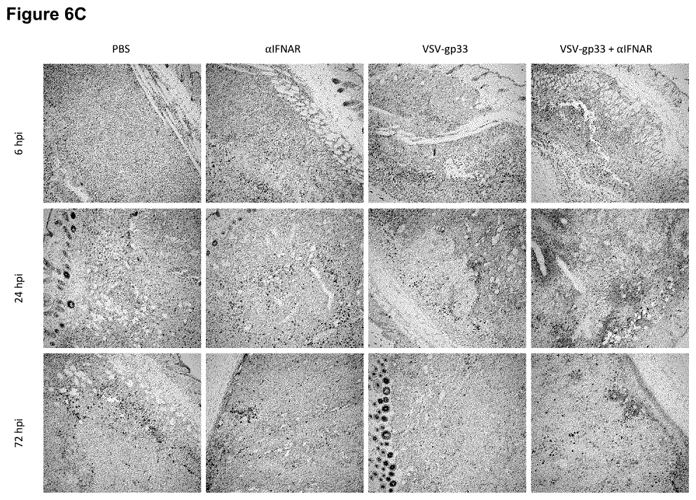

T1IFN has a pronounced role in inhibiting virus replication and spread, an effect to which the ΔM51 mutant VSV virus vector used here is particularly susceptible. Thus, it is speculated that the improved T cell responses observed with IFNAR blockade may be a result of enhanced virus replication leading to increased or prolonged presentation of the gp33 antigen from the virus genome. The effect of IFNAR blockade on virus replication was first characterized using luminescence analysis of mice treated with VSV-SIINFEKL-Luc. Shown in FIG. 6A and FIG. 6B, the intensity and distribution of luminescence signals from infected mice was altered by IFNAR treatment, peaking at 24 hours post infection (hpi) with IFNAR blockade as opposed to 5 hpi with virus alone. Additionally, IFNAR blockade broadened the distribution of luminescence, and this was true with respect to the tumor as well as at distal locations that may represent several lymph nodes, including inguinal, axillary and cervical lymph nodes (white arrows in FIG. 6B). The tail also showed increase luminescence but that is likely a result of endothelial cell infection at the site of injection and may not hold relevance for the therapeutic outcome.

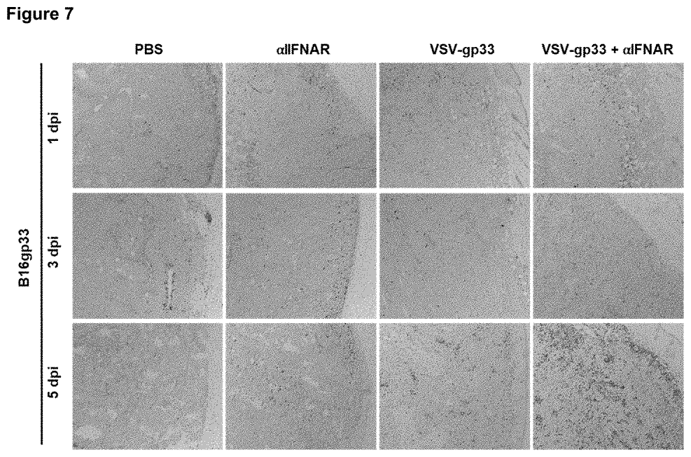

Additional staining was performed on infected tissues using a polyclonal antiserum raised against VSV. As expected, tumor tissues from mice treated with PBS or IFNAR blockade alone showed no staining, confirming the specificity of this antiserum (FIG. 6C). VSV-gp33 alone treated tissues showed only minimal and diffuse staining at 5 hpi and 24 hpi, correlating well with the luciferase results (FIG. 6C). However, intense staining was observed in tumor tissues from mice treated with VSV-gp33+IFNAR blockade, peaking at 24 hpi before waning somewhat by 72 hpi and only minimal diffuse staining at early time points (5 hpi) (FIG. 6C). Staining appears to be localized around vessels in the tumor, as would be expected since the virus was given intravenously (FIG. 6C). Interestingly, staining of treated tumors shows an increased infiltration of the tumor by CD8⁺ cells, in a pattern matching the enhanced T cell responses and virus staining (FIG. 7). VSV vaccination alone also induces infiltration, but to a lesser extent, so it is unclear whether IFNAR blockade augments T cell infiltration or if its effect on the response simply increases the number of cells available for recruitment to the tumor (FIG. 7).

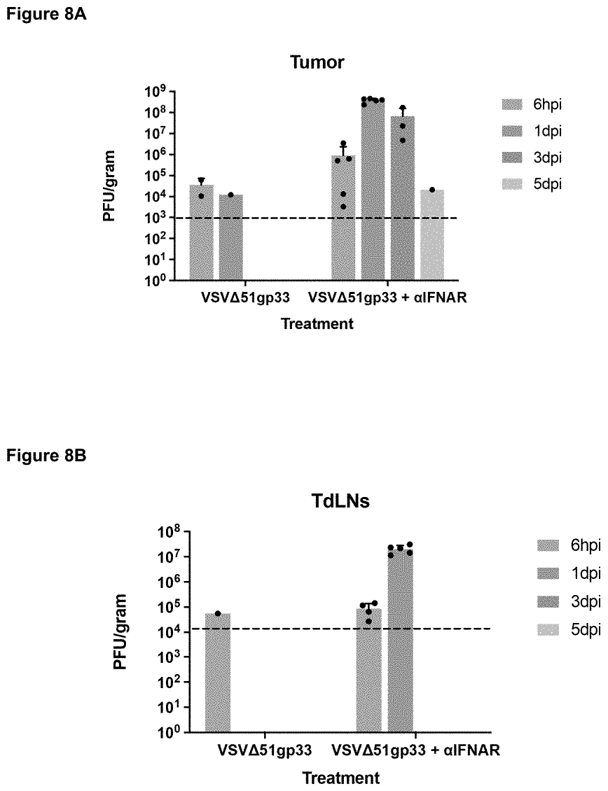

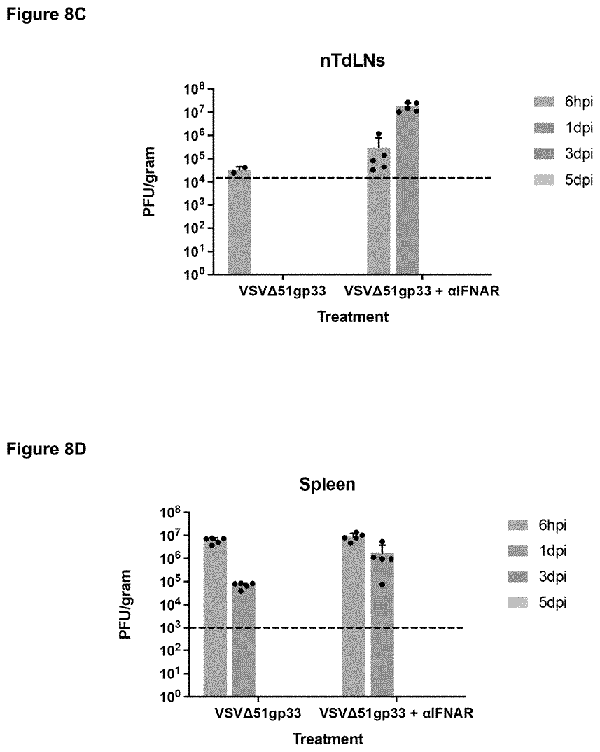

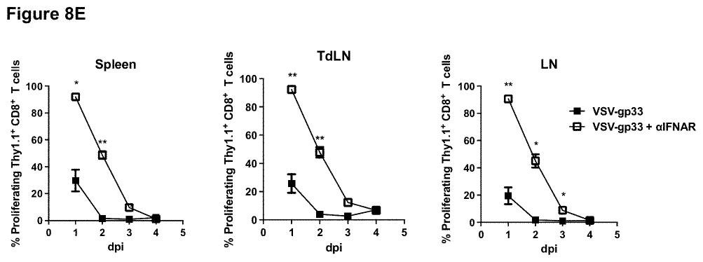

To further characterize and quantify virus replication in the tumor and lymphoid tissues of treated mice a viral plaque assay of tissue homogenates was employed. Plaque counts were significantly increased by IFNAR treatment in tumor tissues and lymph nodes, irrespective of their location relative to the tumor (FIG. 8A-C). As well and similar to what was seen by luminescence and VSV antiserum staining, the peak of plaque counts was shifted to one dpi in tissues from IFNAR treated mice compared to the peak at six hpi observed in virus alone treated tumor tissues and lymph nodes (FIG. 8A to FIG. 8C). Surprisingly, a significant effect was not detected on plaque numbers from spleen homogenates with similar numbers observed from mice treated with virus alone (FIG. 8D).

How IFNAR blockade and its associated effects on VSV replication influenced antigen presentation derived from virus infection was next examined. Transferring proliferation dye labelled naïve P14 T cells at various time points after treatment and assessing dye dilution to infer proliferation as induced by antigen presentation, an increased level and short-term extension in presentation after IFNAR blockade was able to be detected. VSV-gp33 alone supported antigenic stimulation of 20 to 40% of transferred P14 cells one day following vaccination, as determined by dilution of the proliferation dye to indicate at least one division, with no significant proliferation observed at time points assessed thereafter (FIG. 8E). On the other hand, IFNAR antibody treatment augmented the number of transferred T cells showing proliferation one day after treatment with VSV-gp33 to ˜90% and extended the window for antigenic stimulation by 48 hours with observable proliferation in ˜50% and ˜10% of transferred cells at 2 and 3 dpi, respectively (FIG. 8E). In all cases, cells extracted from the spleen and distal lymph nodes showed similar patterns to cells extracted from tumor draining lymph nodes, indicating that presentation was mainly mediated by virus infection and not by cross-presentation of tumor derived antigen (FIG. 8E).

Tumor-Primed T Cells Experience Preferential Increase in Response to Vaccination Driven by IFNAR Blockade

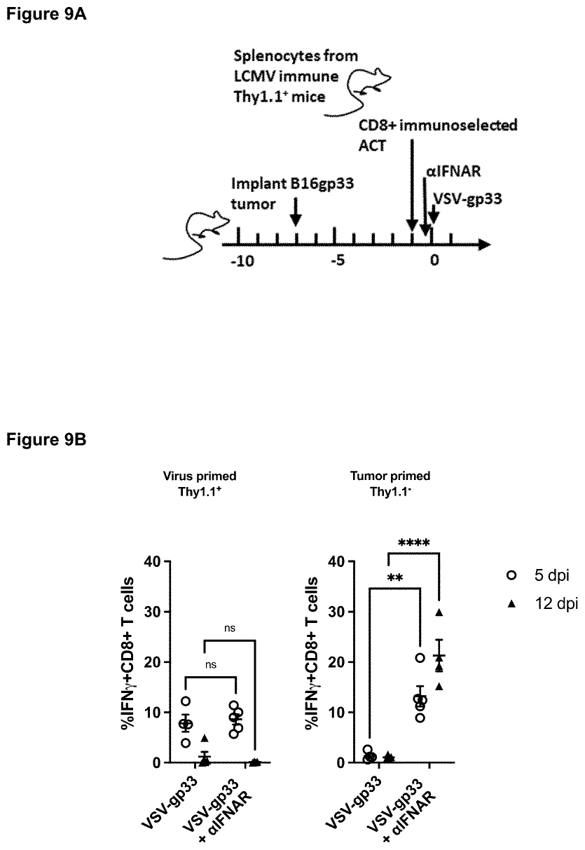

Given that these observations contrast previous reports showing no effect of T1IFN signaling on secondary T cell responses, it was hypothesized that IFNAR blockade mediated augmentation is specific to tumor-primed cells, such as those dominating the response to VSV vaccination. Therefore, in order to simultaneously assess the effect of IFNAR blockade on classically primed T cells and the tumor-primed T cells, purified T cells from Thy1.1⁺ mice previously infected with LCMV were transferred into B16gp33 tumor bearing mice before vaccination (FIG. 9A). Using the Thy1.1 congenic marker to differentiate LCMV-primed T cells and tumor-primed T cells, the response of these distinct subsets to stimulation in the same mouse was assessed and confounding factors, such as enhanced oncolysis and antigen presentation, were eliminated. Responses in this model still showed enhanced magnitudes of gp33-reactive T cells in mice receiving IFNAR blockade but this increase was primarily derived from the Thy1.1⁻ cells, presumably endogenous tumor-primed T cells (FIG. 9B). However, the magnitude of response from LCMV-primed cells (Thy1.1⁺) was not significantly changed by addition of IFNAR blockade to the VSV vaccination treatment (FIG. 9B). These results indicate a specific defect or suppression of tumor-primed T cells to IFNAR signaling in the context of antigenic stimulation that is not shared by T cells primed in the classical method by virus infection.

IFNAR Blockade Enhances and Prolongs Antigen-Specific T Cell Proliferation of Tumor-Primed T Cells

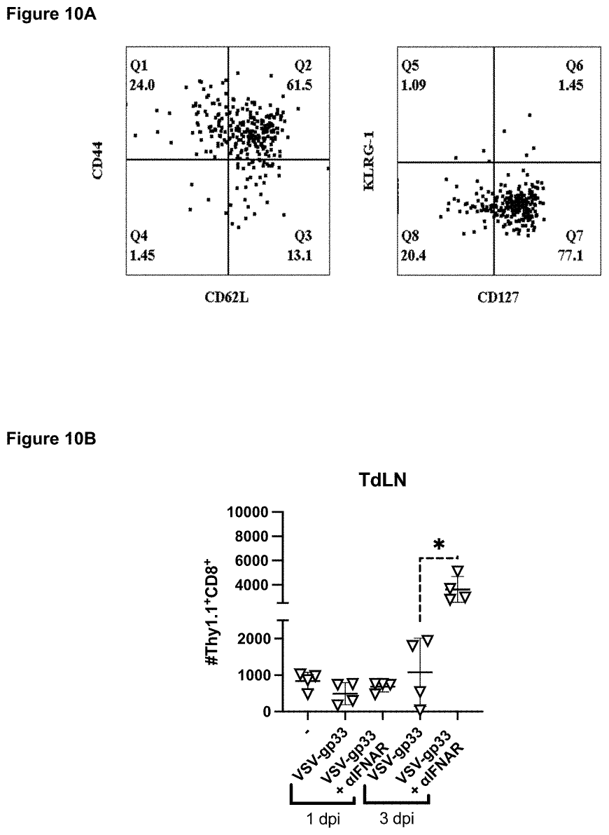

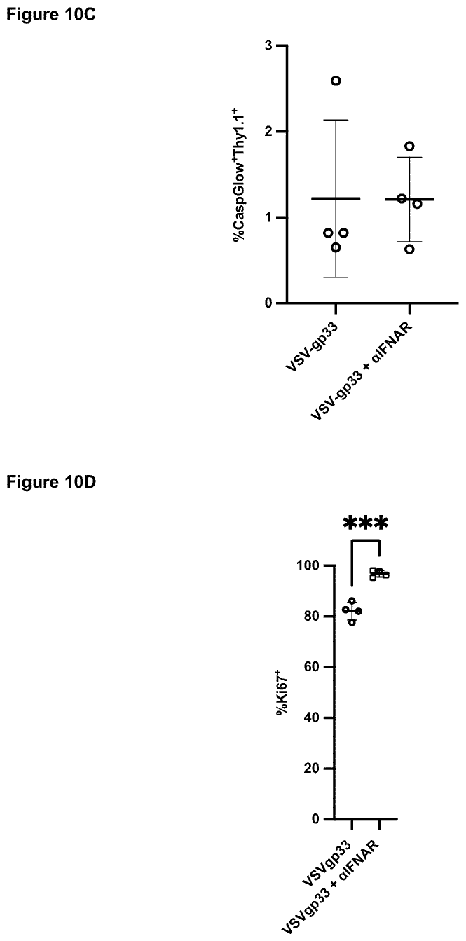

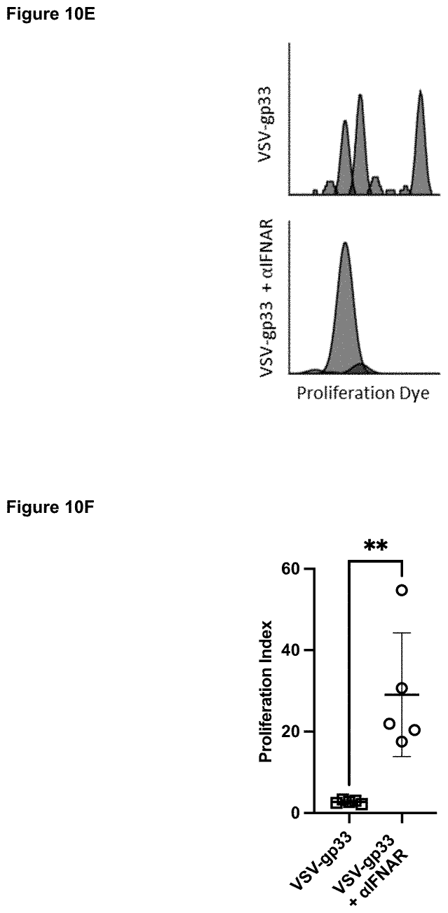

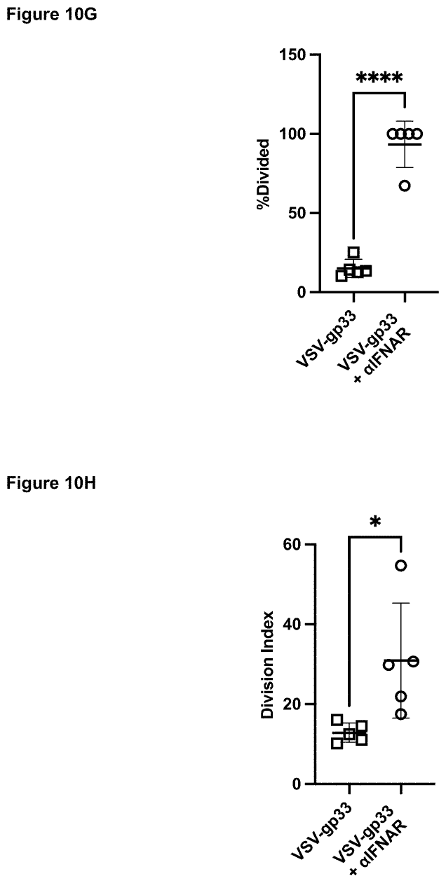

Signaling through IFNAR is most commonly linked to induction of an anti-proliferative, pro-apoptotic state. Thus, blocking IFNAR signaling may be acting to prevent this anti-proliferative, pro-apoptotic state in tumor-primed T cells during VSV vaccination. Accordingly, the effect of IFNAR blockade on the proliferation and/or apoptosis of tumor-primed cells in response to VSV stimulation was investigated. In order to standardize the TCR affinity and generate higher numbers of tumor-primed T cells for analysis, naïve P14 cells transferred one day prior to tumor cell implantation were used to generate tumor-primed P14 T cells. Thy1.1⁺ cells harvested from the tumor-draining lymph nodes seven days after B16gp33 cell injection, a time point at which VSV treatment occurs, had a classical central memory T cell phenotype (FIG. 10A). A uniform high level of CD44 and CD62L expression confirmed that transferred P14 T cells were antigen experienced and lymphoid resident, respectively, and their memory phenotype was further confirmed by high level CD127 and low KLRG1 expression (FIG. 10A). Given that the tumor is the only possible source of gp33 peptide and the early observations with BATF3^{-/-} mice, these cells were presumably generated by cross-presentation of tumor-derived gp33 antigen.

After vaccination, P14Thy1.1⁺ cell numbers were relatively unchanged in the lymph nodes at one dpi compared to before vaccination, indicative of a lack of apoptosis (FIG. 10B). Staining with CaspGlow failed to detect significant apoptosis with or without IFNAR blockade, confirming that apoptosis does not play a role in IFNAR blockade enhanced responses (FIG. 10C). Overall, it appears that VSV vaccination induces very little apoptosis in tumor-primed T cells, leaving no role for IFNAR blockade to play in preventing vaccination induced apoptosis.

Using Ki67 expression levels as marker for progress of these cells through the cell cycle, the effect of IFNAR blockade on proliferation of tumor-primed cells in response to vaccination was next examined. Thy1.1⁺ tumor-primed cells showed increased Ki67 expression levels by flow cytometry staining at three days after vaccination when IFNAR blockade was combined with vaccination (FIG. 10D). Proliferation of tumor-primed P14Thy1.1⁺ cells was further interrogated by proliferation dye labeling. Tumor-primed P14Thy1.1⁺ cells generated and harvested from the tumor-draining lymph node as described above, were labelled with proliferation dye before transfer into tumor free mice followed by vaccination one day later, thus, ensuring that any observed effect was due to the state induced by tumor priming and did not require the presence of a tumor during vaccination. Thy1.1⁺ cells recovered five days after vaccination showed proliferation dye dilution, which was significantly augmented by IFNAR blockade (FIG. 10E). A proliferation curve was fitted and subsequent analysis demonstrated an increased proliferation index in cells from mice receiving IFNAR blockade in combination with VSV vaccination compared to those receiving VSV vaccination alone (FIG. 10F). While differences in proliferation index were partially driven by the fact that the majority of cells in the VSV alone group failed to divide (FIG. 10G), a significant difference was still observable when focusing analysis only on divided cells with the division index (FIG. 10H). Thus, it appears that tumor-primed cells are highly sensitive to the anti-proliferative effects of IFN signaling and IFNAR blockade during vaccination protects from this effect to augment T cell proliferation in response to vaccination and foster the full magnitude of response from these cells.

IFNAR Blockade Regulates the PD-1 PD-L1 Signaling Axis

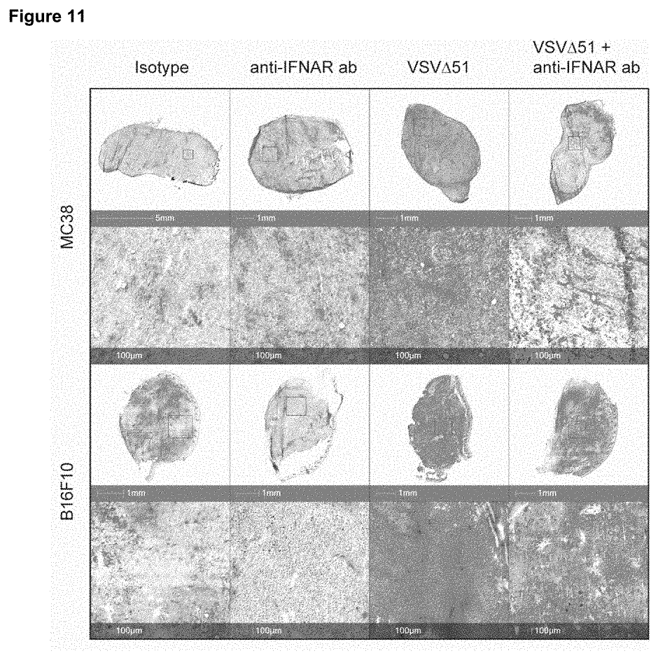

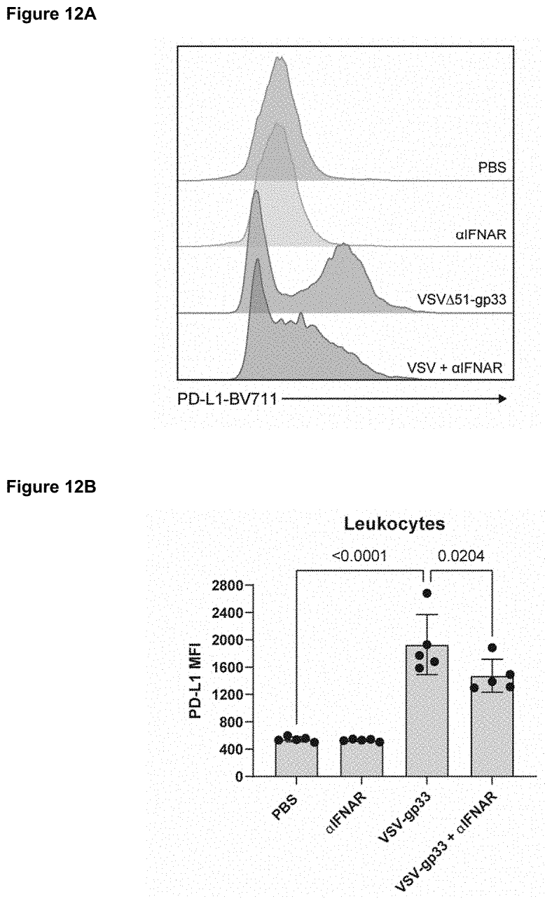

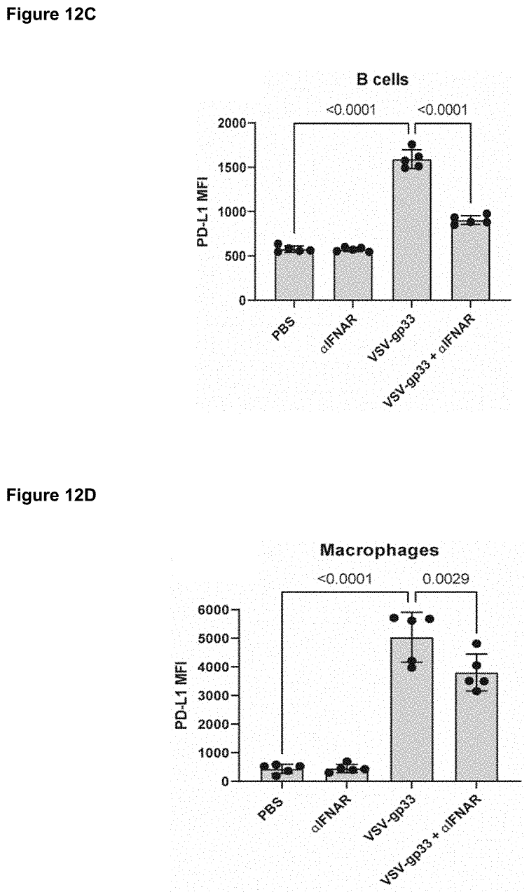

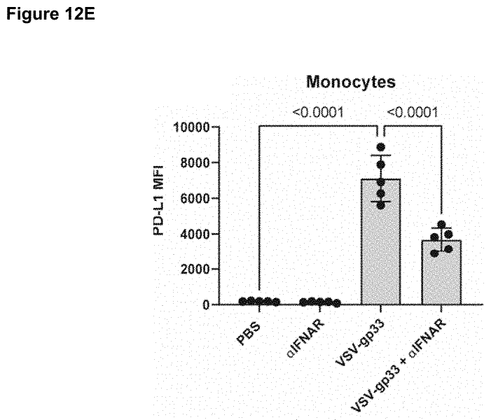

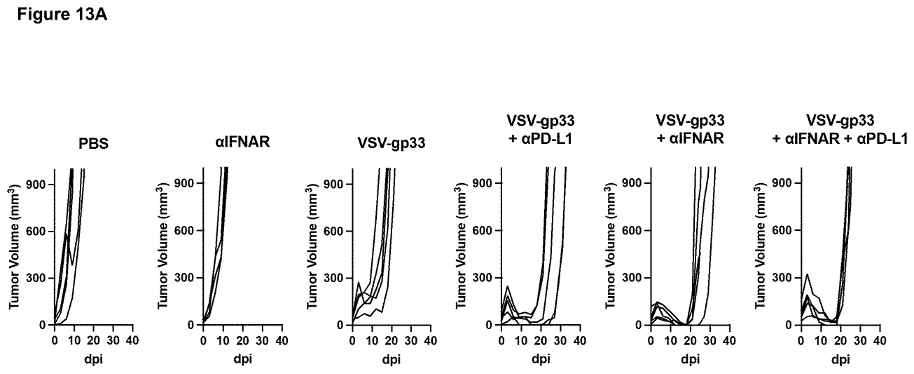

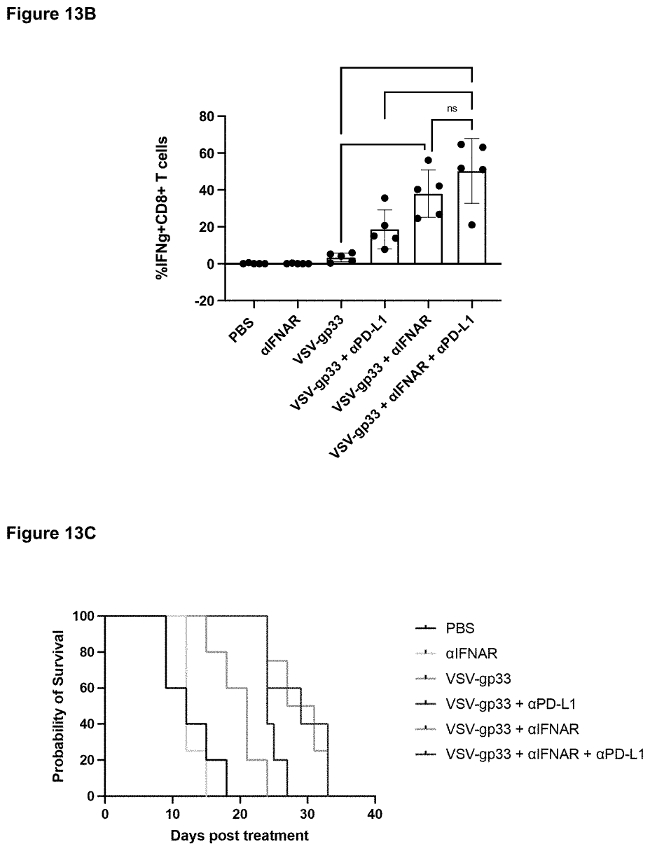

Given the pronounced effect of IFNAR blockade on the magnitude of vaccine induced CD8 T cell responses, augmentation of the killing effect of those responses when used in combination with checkpoint blockade therapies was further characterized. Interferon signaling has previously been shown to modulate the PD-1/PD-L1 axis¹⁸, so the effect of IFNAR blockade on expression of PD-1 and PD-L1 was first examined. As expected, staining of both MC38 and B16 tumors from mice treated with the vaccine alone showed a dramatic increase in PD-L1 staining compared to PBS and IFNAR blockade alone (FIG. 11). However, IFNAR blockade decreased PD-L1 staining, in the context of VSV treatment (FIG. 11). Furthermore, this same pattern on immune cells as a whole was observed (total leukocytes in FIG. 12A and FIG. 12B), although there were some minor variations as to the degree of difference between treatments in more specific immune subsets (FIG. 12C to FIG. 12E). Thus, by inhibiting or limiting upregulation of PD-1 and PD-L1, IFNAR blockade may be also preventing the detrimental effects of the PD-1/PD-L1 signaling axis on T cell cytotoxicity at the tumor. Therefore, the effect of PD-L1 blockade and IFNAR blockade in the context of VSV vaccination of B16gp33 tumor bearing mice was compared. PD-L1 blockade was able to augment both the magnitude of response and the anti-tumor effect of VSV vaccination but failed to reach an equivalent level to IFNAR blockade+VSVgp33 (FIG. 13A and FIG. 13B). In addition, combination of PD-L1 and IFNAR blockade with VSV vaccination (VSV-gp33+αIFNAR+αPD-L1), failed to show any improvement in tumor regression or survival compared to when either blockade therapy was used singularly with VSV vaccination (VSV-gp33+αIFNAR or VSV-gp33+αPD-L1), which implies a redundant function of these blockade therapies (FIG. 13A and FIG. 13C). Thus, it appears that IFNAR blockade also modulates the PD-1/PD-L1 axis, replacing the function of PD-1/PD-L1 CIB therapy by targeting a higher mechanistic point of this pathway.

IFNAR Blockade Synergizes with CTLA-4 Blockade

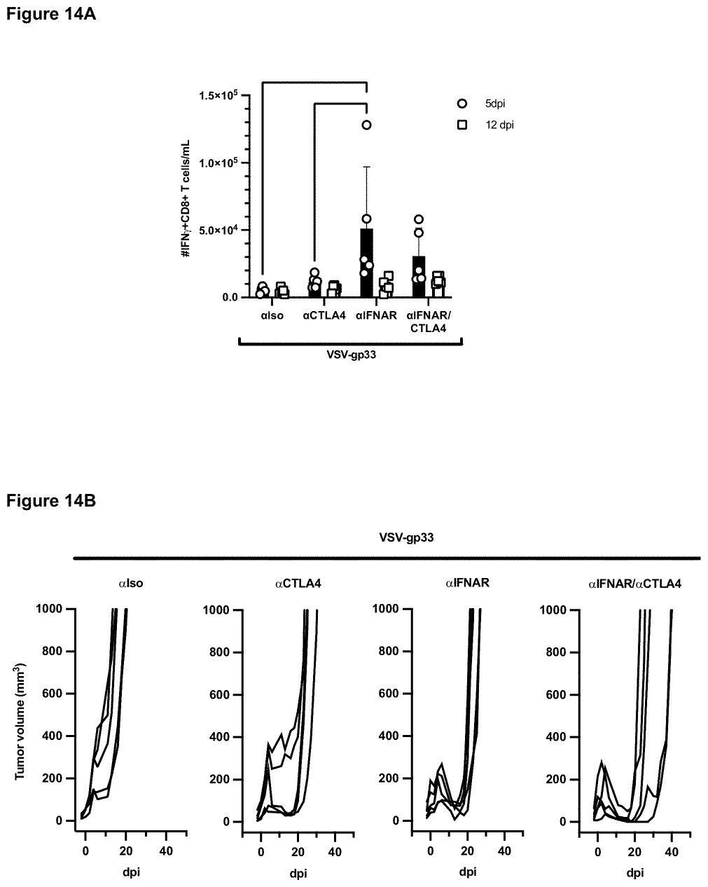

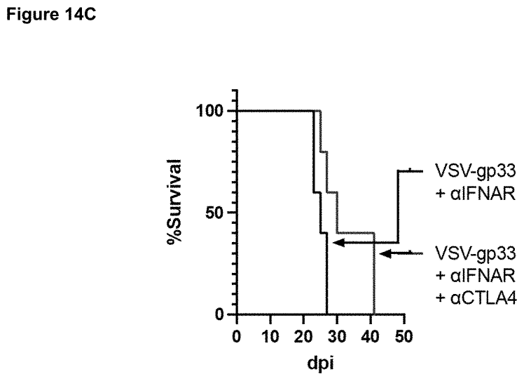

An increase in the total number of CD8 T cells in IFNAR blockade treated vaccinated mice which translates to a shift in the relative ratio of CD8 to CD4 T cells was previously noted (FIG. 3C). Certain clones of CTLA-4 blockade antibodies have been shown to create the same effect via depletion of FoxP3+CD4+T reg cells⁴⁰ so it was postulated that IFNAR blockade may also perform redundant function with CTLA-4 antibody treatment. The combined treatment CTLA-4 antibody with IFNAR blockade and VSV-gp33 vaccination was then tested. CTLA-4 antibody co-treatment generally increased the magnitude of T cell responses induced by VSV-gp33 vaccination but failed to reach significant difference compared to vaccination alone (FIG. 14A). IFNAR blockade again induced an increased response (FIG. 14A) which was correlated with enhanced tumor regression and prolonged survival (FIG. 14B and FIG. 14C). Regression in some of the CTLA-4 treated mice (3 of 5) followed a similar pattern to IFNAR blockade treated mice, demonstrating a potent effect of CTLA-4 treatment in combination with VSV vaccination, but was ultimately less consistent than combination with IFNAR blockade (FIG. 14B). Dual combination of VSV vaccination with both IFNAR and CTLA-4 blockade showed no additional effect on the magnitude of gp33-specific T cell responses compared to when either antibody was used with vaccination alone (FIG. 14A). However, survival was affected in two of five mice, with one showing a delay in relapse and the other showing what appears to be a minor secondary regression amid initial tumor relapse (FIG. 14B). A strong induction of antigen spreading responses during VSV vaccination has been previously observed, generating immunity against a broad profile of tumor antigens beyond the vaccine encoded antigen²⁷. Since it is known that relapse in this model is mediated by antigen loss variant cells, this secondary regression likely represents an antigen spreading response developed against an antigen other than gp33 catalyzed by CTLA-4 treatment. Thus, it is likely that IFNAR and CTLA-4 blockade in combination with VSV vaccination transiently suppressed tumor relapse by augmenting antigen-spreaded responses to facilitate elimination of antigen-negative variant cells.

While the present disclosure has been described with reference to examples, it is to be understood that the scope of the claims should not be limited by the embodiments set forth in the examples, but should be given the broadest interpretation consistent with the description as a whole.

All publications, patents and patent applications are herein incorporated by reference in their entirety to the same extent as if each individual publication, patent or patent application was specifically and individually indicated to be incorporated by reference in its entirety. Where a term in the present disclosure is found to be defined differently in a document incorporated herein by reference, the definition provided herein is to serve as the definition for the term.

### REFERENCES

1. Kantoff, P. W. et al. Sipuleucel-T Immunotherapy for Castration-Resistant Prostate Cancer. http://dx.doi.org 10.1056INEJMoa1001294 363, 411-422 (2010).
2. Chen, D. S. & Mellman, I. Elements of cancer immunity and the cancer-immune set point. Nature 2017 541:7637 541, 321-330 (2017).
3. Melssen, M. M. et al. A multipeptide vaccine plus toll-like receptor agonists LPS or polyICLC in combination with incomplete Freund's adjuvant in melanoma patients. Journal for ImmunoTherapy of Cancer 2019 7:1 7, 1-13 (2019).
4. Gresser, I. & Bourali, C. Exogenous interferon and inducers of interferon in the treatment of balb/c mice inoculated with RC19 tumour cells. Nature 223, 844-845 (1969).
5. Audsley, K. M. et al. IFNβ Is a Potent Adjuvant for Cancer Vaccination Strategies. Front Immunol 0, 3613 (2021).
6. Castro, F., Cardoso, A. P., Gongalves, R. M., Serre, K. & Oliveira, M. J. Interferon-gamma at the crossroads of tumor immune surveillance or evasion. Front Immunol 9, 847 (2018).
7. Katze, M. G., He, Y. & Gale, M. Viruses and interferon: a fight for supremacy. Nature Reviews Immunology 2002 2:9 2, 675-687 (2002).
8. Lee, A. J. & Ashkar, A. A. The Dual Nature of Type I and Type II Interferons. Front Immunol 0, 2061 (2018).
9. Krummel, M. F. et al. Paracrine costimulation of IFN-γ signaling by integrins modulates CD8 T cell differentiation. Proceedings of the National Academy of Sciences 115, 11585-11590 (2018).
10. A, L. B. et al. Direct stimulation of T cells by type I IFN enhances the CD8+ T cell response during cross-priming. J Immunol 176, 4682-4689 (2006).
11. A Alloatti, F. K. J. M. S. A. Dendritic cell maturation and cross-presentation: timing matters! Immunol. Rev. 272, 97-108 (2016).
12. Melero, I. et al. Strict Requirement for Vector-Induced Type I Interferon in Efficacious Antitumor Responses to Virally Encoded IL12. Cancer Res 75, 497-507 (2015).
13. Curtsinger, J M., Lins, D C. & Mescher, M F. Signal 3 determines tolerance versus full activation of naive CD8 T cells: Dissociating proliferation and development of effector function. J Exp Med 197, 1141-1151 (2003).
14. CL, W. et al. Expression of IFN-beta enhances both efficacy and safety of oncolytic vesicular stomatitis virus for therapy of mesothelioma. Cancer Res 69, 7713-7720 (2009).
15. Hosking, M. P., Flynn, C. T. & Whitton, J. L. Type I IFN Signaling Is Dispensable during Secondary Viral Infection. PLoS Pathog 12, e1005861 (2016).
16. Jacquelot, N. et al. Sustained Type I interferon signaling as a mechanism of resistance to PD-1 blockade. Cell Research 2019 29:10 29, 846-861 (2019).
17. Benci, J. L. et al. Tumor Interferon Signaling Regulates a Multigenic Resistance Program to Immune Checkpoint Blockade. Cell 167, 1540-1554.e12 (2016).
18. Garcia-Diaz, A. et al. Interferon Receptor Signaling Pathways Regulating PD-L1 and PD-L2 Expression. Cell Rep 19, 1189 (2017).
19. Evgin, L. et al. Oncolytic virus-derived type I interferon restricts CAR T cell therapy. Nature Communications 2020 11:1 11, 1-15 (2020).
20. Chabanon, R. M. et al. Mutational landscape and sensitivity to immune checkpoint blockers. Clinical Cancer Research 22, 4309-21 (2016).
21. Snyder, A. et al. Genetic Basis for Clinical Response to CTLA-4 Blockade in Melanoma. New England Journal of Medicine 371, 2189-2199 (2014).
22. McGranahan, N. et al. Clonal neoantigens elicit T cell immunoreactivity and sensitivity to immune checkpoint blockade. Science (1979) 351, 1463-9 (2016).
23. Hugo, W. et al. Genomic and Transcriptomic Features of Response to Anti-PD-1 Therapy in Metastatic Melanoma. Cell 165, 35-44 (2016).
24. Philip, M. et al. Chromatin states define tumour-specific T cell dysfunction and reprogramming. Nature 2017 545:7655 545, 452-456 (2017).
25. Houot, R., Schultz, L. M., Marabelle, A. & Kohrt, H. T-cell-based Immunotherapy: Adoptive Cell Transfer and Checkpoint Inhibition. Cancer Immunol Res 3, 1115-1122 (2015).
26. Joyce, J. A. & Fearon, D. T. T cell exclusion, immune privilege, and the tumor microenvironment. Science (1979) 348, 74-80 (2015).
27. Walsh, S. R. et al. Endogenous T cells prevent tumor immune escape following adoptive T cell therapy. Journal of Clinical Investigation 129, (2019).
28. Walsh, S. R. et al. Type I IFN blockade uncouples immunotherapy-induced antitumor immunity and autoimmune toxicity. Journal of Clinical Investigation 129, (2019).
29. Thoidingjam, L. K. et al. Small molecule inhibitors of interferon-induced JAK-STAT signalling. Angewandte Chemie Int. Ed. 61, e202205231 (2022).
30. Prevost-Blondel, A. et al. Tumor-infiltrating lymphocytes exhibiting high ex vivo cytolytic activity fail to prevent murine melanoma tumor growth in vivo. J Immunol vol. 161 2187-2194 Preprint at http://www.ncbi.nlm.nih.gov/entrez/query.fcgi?cmd=Retrieve&db=PubMed&d opt=Citation&list_uids=9725210 (1998).
31. Hayer, A. et al. Engulfed cadherin fingers are polarized junctional structures between collectively migrating endothelial cells. Nat Cell Biol 18, 1311-1323 (2016).
32. Diallo, J. S., Vaha-Koskela, M., le Boeuf, F. & Bell, J. Propagation, purification, and in vivo testing of oncolytic vesicular stomatitis virus strains. Methods Mol Biol 797, 127-140 (2012).
33. Pol, J. G. et al. Maraba virus as a potent oncolytic vaccine vector. Molecular therapy 22, 420-9 (2014).
34. Bridle, B. W. et al. Privileged Antigen Presentation in Splenic B Cell Follicles Maximizes T Cell Responses in Prime-Boost Vaccination. The Journal of Immunology 196, 4587-4595 (2016).
35. Stojdl, D. F. et al. VSV strains with defects in their ability to shutdown innate immunity are potent systemic anti-cancer agents. Cancer Cell 4, 263-275 (2003).
36. Boyd, A. et al. Pathogen-specific T cell polyfunctionality is a correlate of T cell efficacy and immune protection. PLoS One 10, (2015).
37. Imai, N. et al. Glucocorticoid-induced tumor necrosis factor receptor stimulation enhances the multifunctionality of adoptively transferred tumor antigen-specific CD8+ T cells with tumor regression. Cancer Sci 100, 1317-1325 (2009).
38. Nguyen, A. et al. HDACi Delivery Reprograms Tumor-Infiltrating Myeloid Cells to Eliminate Antigen-Loss Variants. Cell Rep (2018) doi:10.1016/j.celrep.2018.06.040.
39. Bridle, B. W. et al. Potentiating cancer immunotherapy using an oncolytic virus. Molecular therapy 18, 1430-9 (2010).
40. Wei, S. C., Duffy, C. R. & Allison, J. P. Fundamental Mechanisms of Immune Checkpoint Blockade Therapy. Cancer Discov 8, 1069-1086 (2018).

What is claimed is:

1. A method for stimulating tumor antigen-specific immune responses comprising administering an inhibitor of interferon (IFN) signaling and a cancer vaccine comprising a tumor antigen to a subject in need thereof; wherein the inhibitor of IFN is administered before the cancer vaccine.

2. The method of claim 1 for treating cancer in the subject.

3. The method of claim 2, wherein the cancer is selected from the group consisting of melanoma, sarcoma, lymphoma, carcinoma, brain cancer, breast cancer, liver cancer, lung cancer, kidney cancer, pancreatic cancer, esophageal cancer, stomach cancer, colon cancer, colorectal cancer, bladder cancer, prostate cancer and leukemia.

4. The method of claim 1, wherein the inhibitor is administered from about 5 minutes to about 48 hours before the cancer vaccine is administered.

5. The method of claim 1, wherein the inhibitor is administered from about 30 minutes to about 24 hours before the cancer vaccine is administered.

6. The method of claim 1, wherein the inhibitor is administered from about 2 hours to about 4 hours before the cancer vaccine is administered.

7. The method of claim 1, wherein the interferon comprises type 1 interferon (T1IFN) and/or type 2 interferon (T2IFN).

8. The method of claim 1, wherein the inhibitor comprises an antibody, or fragment thereof, that specifically binds and neutralizes IFNα and/or IFNβ.

9. The method of claim 1, wherein the inhibitor comprises an antibody, or fragment thereof, that specifically binds and blocks a T1IFN receptor.

10. The method of claim 1, wherein the inhibitor is an antibody, or fragment thereof, that binds and neutralizes IFNγ.

11. The method of claim 1, wherein the inhibitor is an antibody, or fragment thereof, that specifically binds and blocks a T2IFN receptor.

12. The method of claim 1, wherein the cancer vaccine comprises an oncolytic virus that expresses a tumor antigen.

13. The method of claim 12, wherein the oncolytic virus is selected from the group consisting of Herpesviridae, Rhabdoviridae, Picornaviradae, Reoviridae, Togaviridae, Adenoviridae, Paramyxoviridae, and Poxviridae.

14. The method of claim 12, wherein the oncolytic virus is a rhabdovirus.

15. The method of claim 14, wherein the rhabdovirus is a recombinant or wildtype vesicular stomatitis virus.

16. The method of claim 1, wherein the cancer vaccine and/or the inhibitor is administered by injection.

17. The method of claim 1, wherein the tumor antigen is a tumor-associated antigen and/or a tumor-specific antigen of the cancer.

18. The method of claim 17, wherein the tumor-associated antigen is selected from the group consisting of alphafetoprotein (AFP), carcinoembryonic antigen (CEA), CA 125, Her2, dopachrome tautomerase (DCT), GP100, Melan-A/MART-1, MAGE proteins, BAGE proteins, GAGE proteins, NY-ESO1, WT-1, survivin, tyrosinase, SSX2, Cyclin-A1, KIF20A, MUC5AC, Meloe, Lengsin, Kallikrein 4, IGF2B3, glypican-3, HPV E6 and HPV E7.

19. The method of claim 1, wherein the method further comprises administering adoptive cell transfer (ACT) cells and/or a checkpoint inhibitor.

20. A kit comprising an inhibitor of interferon (IFN) signaling, a cancer vaccine comprising a tumor antigen, and instructions for use.
